(19) United States
(12) Patent Application Publication
(10) Pub. No.: US 2025/0282991 A1
(43) Pub. Date: Sep. 11, 2025

(54) Light conversion agent, a light conversion adhesive film composition, a light conversion adhesive film, and a photovoltaic module

(71) Applicant: HANGZHOU FIRST APPLIED MATERIAL CO., LTD., Hangzhou (CN)

(72) Inventors: Jin LIU, Hangzhou (CN); Qiwei ZHU, Hangzhou (CN); Mengjuan WEI, Hangzhou (CN); Hongbing HOU, Hangzhou (CN); Guangda ZHOU, Hangzhou (CN)

(21) Appl. No.: 18/622,651

(22) Filed: Mar. 29, 2024

(30) Foreign Application Priority Data
Mar. 11, 2024 (CN) 202410274685.2

(51) Int. Cl. C09K 11/06; C07D 249/22; C09J 7/38; C09J 11/06; C09K 11/02; H10K 30/20

(52) U.S. Cl. CPC C09K 11/06; C07D 249/22; C09J 7/381; C09J 11/06; C09K 11/02; C09J 2203/322; C09J 2301/408; C09J 2301/414; C09J 2423/04; C09K 2211/1007; C09K 2211/1014; C09K 2211/1018; H10K 30/20

(57) ABSTRACT

The present disclosure provides a light conversion agent, a light conversion adhesive film composition, a light conversion adhesive film, and a photovoltaic module. The light conversion agent is a naphthotriazole compound, the naphthotriazole compound has the structural general formula as shown as follows:

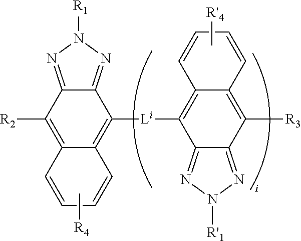

wherein, i is any integer from 0 to 100; R₁ and R′₁ are selected from the substituent of a substituted or unsubstituted C₁-C₂₀ alkyl, etc., and R₂, R₃, R₄ and R′₄ are each independently selected from the substituent of H, or a substituted or unsubstituted C₁-C₂₀ alkyl, etc. The naphthotriazole ring can often exhibit a broader band of absorption. The light conversion adhesive film obtained by using the light conversion adhesive film composition of this disclosure has excellent light stability and high luminous efficiency, which can effectively function in the long term, thereby improving the service life of the photovoltaic devices (such as photovoltaic modules).

### TECHNICAL FIELD

The present disclosure relates to the field of optoelectronic technology, specifically to a light conversion agent, a light conversion adhesive film composition, a light conversion adhesive film, and a photovoltaic module.

### BACKGROUND

Solar energy, as a clean energy that is rich in resources and does not require transportation, is expected to become an alternative energy to replace traditional fossil fuels. Therefore, the development of solar energy acquisition and conversion technologies has gradually become a focus of attention for the country and society. Photovoltaic devices can directly convert solar energy into electricity, which is one of the most effective ways to utilize solar energy. So far, the demand for photovoltaic installed capacity on a global scale is still continuously increasing.

Due to the fact that the vast majority of photovoltaic devices can only effectively utilize the visible of sunlight and near-infrared wavelengths of sunlight, the utilization efficiency of ultraviolet light (wavelength of less than 400 nm) in sunlight is relatively low. Moreover, the presence of ultraviolet light in sunlight can also reduce the service life of photovoltaic devices, especially for heterojunction (HJT) cells that are expected to replace Passivated Emitter and Rear Cells (PERCs) and Tunnel Oxide Passivating Contacts (TOPCon) and become the third-generation solar cells. The use of light conversion adhesive film can not only effectively fully absorb the ultraviolet light in sunlight, but also can avoid its damage to the service life of photovoltaic devices. At the same time, ultraviolet light can be converted into usable visible light, thereby improving the photoelectric conversion efficiency of photovoltaic devices.

Especially with the development of HJT cell technology, the importance of light conversion adhesive film has also increased. Due to the fact that for HJT cells, in order to achieve higher photoelectric conversion efficiency, some sacrifices in terms of UV stability have to be made for the cell modules. The daily use scenarios of photovoltaic devices obviously inevitably require exposure to strong ultraviolet radiation. Therefore, using a light conversion adhesive film with high ultraviolet absorption and intense luminescence for modular package can not only effectively absorb the ultraviolet light from sunlight to avoid module damage, but also the absorbed ultraviolet light can be converted into visible light to improve the utilization efficiency of the cells for sunlight and achieve the gain effect. In summary, developing a high-performance light conversion adhesive film is of great significance for the development of HJT cells.

The core of the light conversion adhesive film technology lies in the selection of the light conversion agents. At present, common types of light conversion agents include: organic small molecules, polymers, quantum dots, organometallic complexes, etc. However, overall consideration of various factors such as cost, dispersibility, light conversion property, visible light transmittance, etc., organic small molecular light conversion agents (compared to inorganic light conversion agents) have the greatest potential for application. However, the performance of organic light conversion agents in terms of light stability is poor currently. Under the ultraviolet radiation, their light conversion ability is prone to significant decay and yellowing, which is often an unacceptable defect for adhesive film products. In recent years, a series of organic small molecular light conversion agents have been developed based on triazole structures, which exhibit high light stability. The research results indicate that the shortcomings of organic small molecular light conversion agents in terms of light stability are not insurmountable. However, there is still an urgent need to develop a light conversion adhesive film with higher photoelectric conversion efficiency, better stability, and longer service life.

### SUMMARY

The main object of the present disclosure is to provide a light conversion agent, a light conversion adhesive film composition, a light conversion adhesive film, and a photovoltaic module, in order to solve the problems of low photoelectric conversion efficiency, poor stability, and short service life of traditional light conversion adhesive films in the prior art.

In order to achieve the above object, according to one aspect of the present disclosure, a light conversion agent is provided, the light conversion agent is a naphthotriazole compound, and the naphthotriazole compound has the structural general formula as shown as follows:

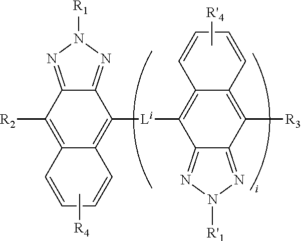

Wherein, i is any integer from 0 to 100; Lⁱ is independently selected from any one of a substituted or unsubstituted alkylene, a substituted or unsubstituted alkenylene, a substituted or unsubstituted arylene, and a substituted or unsubstituted heteroarylene; R₁ and R′₁ are each independently selected from any one of a substituted or unsubstituted C₁-C₂₀ alkyl, a substituted or unsubstituted C₂-C₂₀ heteroalkyl, a substituted or unsubstituted C₂-C₂₀ alkenyl, a substituted or unsubstituted C₆-C₄₀ aryl, a substituted or unsubstituted C₄-C₄₀ heteraryl, a substituted or unsubstituted C₂-C₂₀ ester group, a C₁-C₂₀ hydrocarbyl substituted or unsubstituted amino, a C₆-C₃₀ aryl substituted or unsubstituted amino, a C₁-C₂₀ hydrocarbyl substituted or unsubstituted amido, a substituted or unsubstituted C₃-C₂₀ cyclic amido, a substituted or unsubstituted C₃-C₂₀ cyclic imide group, a C₁-C₂₀ hydrocarbyl substituted or unsubstituted carboxyl, a C₁-C₂₀ hydrocarbylsubstituted or unsubstituted carbonyl, and a C₁-C₂₀ hydrocarbyl substituted or unsubstituted hydroxyl; R₂, R₃, R₄ and R′₄ are each independently selected from any one of H, a substituted or unsubstituted C₁-C₂₀ alkyl, a substituted or unsubstituted C₂-C₂₀ heteroalkyl, a substituted or unsubstituted C₂-C₂₀ alkenyl, a substituted or unsubstituted C₆-C₄₀ aryl, a substituted or unsubstituted C₄-C₄₀ heteraryl, a substituted or unsubstituted C₂-C₂₀ ester group, a C₁-C₂₀ hydrocarbyl substituted or unsubstituted amino, a C₆-C₃₀ aryl substituted or unsubstituted amino, a C₁-C₂₀ hydrocarbyl substituted or unsubstituted amido, a substituted or unsubstituted C₃-C₂₀ cyclic amido, a substituted or unsubstituted C₃-C₂₀ cyclic imide group, a C₁-C₂₀ hydrocarbyl substituted or unsubstituted carboxyl, a C₁-C₂₀ hydrocarbyl substituted or unsubstituted carbonyl, and a C₁-C₂₀ hydrocarbyl substituted or unsubstituted hydroxyl; wherein, the heteroatoms in heteroalkyl, heteroaryl, and heteroarylene groups are selected from any one or more of N, O, and S; and one or more methylene groups in the R₁, R′₁, R₂, R₃, R₄ and R′₄ are optionally substituted by —O— or —S—.

Further, when the above R₁, R₂, R₃ and R₄ carry a substituent, the substituent is selected from any one or more of C₁-C₆ alkyl, C₁-C₆ alkoxyl, C₂-C₆ alkenyl, C₂-C₆ alkynyl, geminally linked cycloalkyl, C₃-C₆ heterocycloalkyl, phenyl, anilino, naphthyl, biphenyl, halogen, hydroxyl, carboxyl, nitro, trifluoromethyl, trifluoromethoxy, cyano, amino, amido, and C₂-C₁₀ ester group; preferably, the substituent is selected from any one or more of methyl, ethyl, propyl, butyl, pentyl, hexyl, methoxy, ethoxy, propoxy, butoxy, pentyloxy, hexyloxy, ethenyl, propenyl, butenyl, pentenyl, hexenyl, ethynyl, propynyl, butynyl, pentynyl, hexynyl, cyclopropyloxy, cyclobutoxy, cyclopentoxy, cyclohexoxy, phenyl, anilino, naphthyl, biphenyl, halogen, hydroxyl, carboxyl, nitro, trifluoromethyl, trifluoromethoxy, cyano, amino, formamido, methyl formate group, ethyl formate group, ethyl propionate group, butyl propionate group, ethyl butyrate group, butyl butyrate group, methyl hexanoate group, methyl heptanoate group, and ethyl caprylate group.

further, the above R₁ is selected from any one of a substituted or unsubstituted C₂-C₁₀ linear alkyl, a substituted or unsubstituted C₃-C₁₅ branched alkyl, a substituted or unsubstituted C₂-C₁₀ heteroalkyl, a substituted or unsubstituted C₂-C₁₀ alkenyl, a substituted or unsubstituted C₂-C₁₀ ester group, a C₁-C₁₀ hydrocarbyl substituted or unsubstituted amino, a C₆-C₂₀ aryl substituted or unsubstituted amino, a C₁-C₁₀ hydrocarbyl substituted or unsubstituted amido, a substituted or unsubstituted C₃-C₁₀ cyclic amido, a substituted or unsubstituted C₃-C₁₀ cyclic imide group, a C₁-C₁₀ hydrocarbyl substituted or unsubstituted carboxyl, a C₁-C₁₀ hydrocarbyl substituted or unsubstituted carbonyl, and a C₁-C₁₀ hydrocarbyl substituted or unsubstituted hydroxyl; the R₂, the R₃ and the R₄ are each independently selected from any one of H, a substituted or unsubstituted C₂-C₁₀ linear alkyl, a substituted or unsubstituted C₃-C₁₅ branched alkyl, a substituted or unsubstituted C₂-C₁₀ heteroalkyl, a substituted or unsubstituted C₂-C₁₀ alkenyl, a substituted or unsubstituted C₆-C₃₀ aryl, a substituted or unsubstituted C₄-C₂₀ heteraryl, a substituted or unsubstituted C₂-C₁₀ ester group, a C₁-C₁₀ hydrocarbyl substituted or unsubstituted amino, a C₆-C₂₀ aryl substituted or unsubstituted amino, a C₁-C₁₀ hydrocarbyl substituted or unsubstituted amido, a substituted or unsubstituted C₃-C₁₀ cyclic amido, a substituted or unsubstituted C₃-C₁₀ cyclic imide group, a C₁-C₁₀ hydrocarbyl substituted or unsubstituted carboxyl, a C₁-C₁₀ hydrocarbyl substituted or unsubstituted carbonyl, and a C₁-C₁₀ hydrocarbyl substituted or unsubstituted hydroxyl; preferably, the R₁, the R₂, the R₃ and the R₄ are each independently selected from any one of the following substituted or unsubstituted substituents: n-hexyl, n-heptyl, n-octyl, n-nonyl, n-decyl, isopropyl, isobutyl, isopentyl, isohexyl, isoheptyl, isooctyl, isononyl, isodecyl, cyclopropyloxy, cyclobutoxy, cyclopentoxy, cyclohexyloxy, hexyloxy, heptyloxy, octyloxy, nonyloxy, decyloxy, ethenyl, propenyl, butenyl, pentenyl, hexenyl, heptenyl, octenyl, nonenyl, decenyl, allyl, phenyl, naphthyl, biphenyl, furyl, thienyl, indolyl, pyridyl, benzofuryl, benzothiophenyl, methyl formate group, ethyl formate group, ethyl propionate group, butyl propionate group, ethyl butyrate group, butyl butyrate group, methyl hexanoate group, methyl heptanoate group, ethyl caprylate group, amino, formamido, acetamido, propionamido, butyramido, pentanamido, cyclopropionamido, cyclobutyramido, cyclopropanoylimino, cyclobutanoylimino, carboxyl, or carbonyl.

Further, the above naphthotriazole compound has the structural general formula as shown as follows:

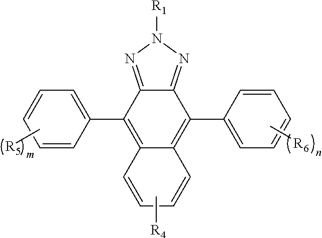

Wherein, m and n are each independently 1, 2, 3, or 4; R₁ is selected from any one of a substituted or unsubstituted C₁-C₁₂ linear alkyl, a substituted or unsubstituted C₃-C₁₀ branched alkyl, a substituted or unsubstituted C₂-C₁₆ alkenyl, a substituted or unsubstituted C₃-C₈ ester group, a C₅-C₈ hydrocarbyl substituted or unsubstituted amino, a C₆-C₁₀ aryl substituted or unsubstituted amino, a C₅-C₈ hydrocarbyl substituted or unsubstituted amido, a substituted or unsubstituted C₅-C₈ cyclic amido, a substituted or unsubstituted C₅-C₈ cyclic imide group, a C₅-C₈ hydrocarbyl substituted or unsubstituted carboxyl, a C₅-C₈ hydrocarbyl substituted or unsubstituted carbonyl, and a C₅-C₈ hydrocarbyl substituted or unsubstituted hydroxyl; preferably, the R₄, the R₅ and the R₆ are each independently selected from any one of H, a substituted or unsubstituted C₁-C₁₂ linear alkyl, a substituted or unsubstituted C₃-C₁₀ branched alkyl, a substituted or unsubstituted C₂-C₁₆ alkenyl, a substituted or unsubstituted C₃-C₈ ester group, a C₅-C₈ hydrocarbyl substituted or unsubstituted amino, a C₆-C₁₀ aryl substituted or unsubstituted amino, a C₅-C₈ hydrocarbyl substituted or unsubstituted amido, a substituted or unsubstituted C₅-C₈ cyclic amido, a substituted or unsubstituted C₅-C₈ cyclic imide group, a C₅-C₈ hydrocarbyl substituted or unsubstituted carboxyl, a C₅-C₈ hydrocarbyl substituted or unsubstituted carbonyl, and a C₅-C₈ hydrocarbyl substituted or unsubstituted hydroxyl; preferably, the R₄ is H; preferably, the R₁, the R₅ and the R₆ are each independently selected from any one or more of a substituted or unsubstituted C₅-C₈ linear alkyl, a substituted or unsubstituted C₆-C₁₀ branched alkyl, a substituted or unsubstituted C₅-C₈ alkenyl, a substituted or unsubstituted C₃-C₈ ester group, and a phenyl substituted or unsubstituted amino, further, preferably, the R₁, the R₅ and the R₆ are each independently selected from any one of

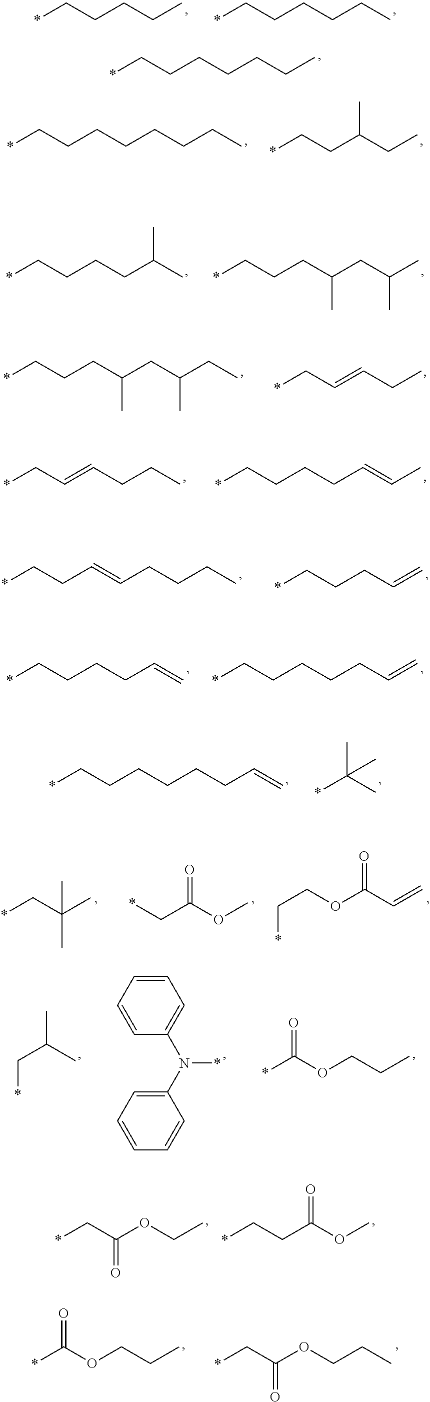

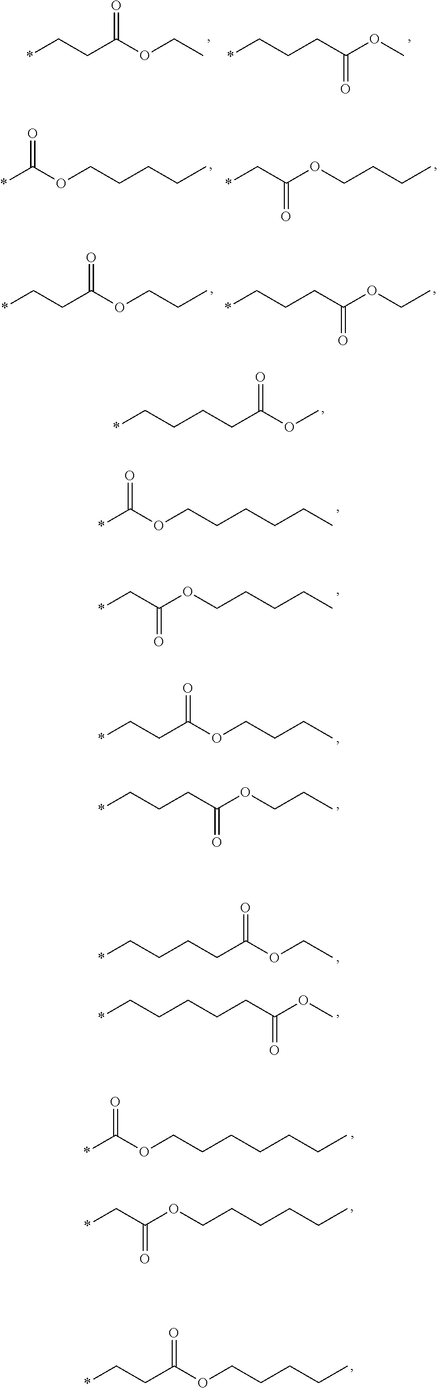

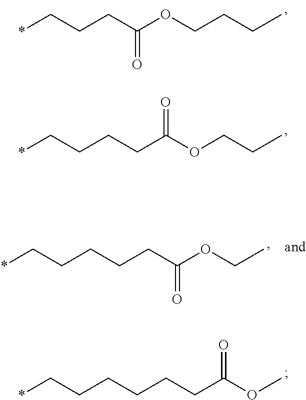 [SMILES: *CCCC(=O)OCCCC ; *CCCCC(=O)OCCC ; *CCCCCC(=O)OCC ; *CCCCCCC(=O)OC]

further, preferably, the R₁ is selected from any one of

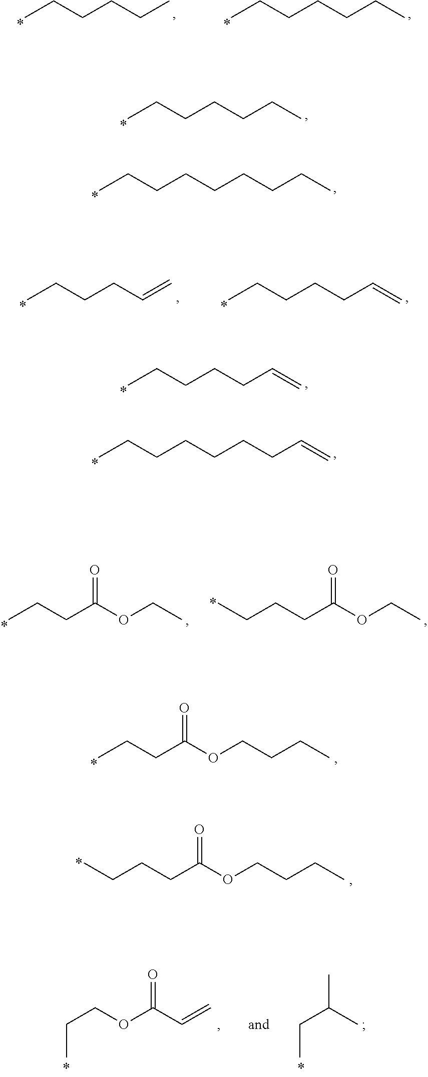

preferably, the R₅ and the R₆ are each independently selected from any one of

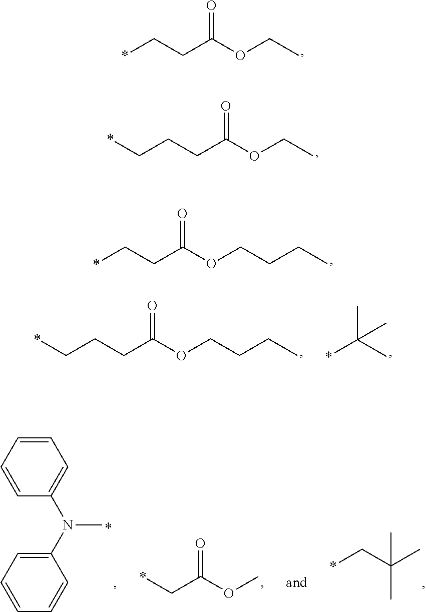

further, preferably, the R₅ and the R₆ are simultaneously any one of

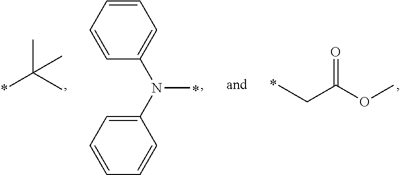

wherein, “*” indicates the attachment position of the substituent on the naphthotriazoles.

According to another aspect of the present disclosure, a light conversion adhesive film composition is provided, in terms of weight percentage, the light conversion adhesive film composition includes 80% to 99.98% of a matrix resin, 0.01% to 10% of a light conversion agent and 0.01% to 10% of an auxiliary agent, wherein the light conversion agent is the aforementioned light conversion agent.

Further, in terms of weight percentage, the light conversion adhesive film composition includes 98% to 99.98% of a matrix resin, 0.01% to 1% of a light conversion agent and 0.01% to 1% of an auxiliary agent; further, preferably, the mass ratio of the light conversion agent to the auxiliary agent is 1:2-500, preferably 1:2-50.

Further, the above matrix resin is selected from any one or more of EVA, PVA, PMMA, POE, and an organic silicon.

Further, the above auxiliary agent includes any one of a main crosslinking agent, an auxiliary crosslinking agent, a silane coupling agent, and an inorganic powder, or a combination of at least two of the above.

According to yet another aspect of the present disclosure, a light conversion adhesive film is provided, which is obtained by mixing and molding an adhesive film composition, and the adhesive film composition is the aforementioned light conversion adhesive film composition.

According to yet another aspect of the present disclosure, a photovoltaic module is provided, comprising a light conversion adhesive film, which is the aforementioned light conversion adhesive film.

Applying the technical solution of the present disclosure, the triazole structure, as a building block of a classic optoelectronic functional material, has very unique electronic properties. Specifically, the three connected N atoms on the five-membered aromatic ring of the triazole structure not only enable the triazole to have both electron donating and electron withdrawing abilities, but also enable the triazole to have a higher electron cloud density compared to imidazole. When the triazole structure is connected with an aromatic ring to form a structure such as benzotriazole or naphthotriazole, the low-density π electron cloud of the benzene ring can effectively alleviate the electron cloud density on the triazole, so that the benzotriazole and naphthotriazole structures become very special electron-rich acceptors. Such electron-rich receptor structure can form an effective conjugated system even when it is connected with some electron donating groups having weaker electron donating abilities, thereby achieving electron transition and recovery from the ground state to the excited state, and radioluminescence. It should be specifically noted that compared to benzotriazole, the naphthotriazole with an additional benzene ring in this disclosure has a larger conjugate plane, which enables the naphthotriazole to have better coordination with respect to both electron donating and electron withdrawing. Therefore, when some auxochrome groups are attached to the periphery of the naphthotriazole ring, the naphthotriazole ring can often exhibit a more red-shifted absorption and emission effect. In addition, an additional benzene ring will inevitably lead to a lower LUMO for naphthotriazole compared to benzotriazole. Therefore, when the peripheral auxochrome groups of the naphthotriazole ring are identical, the naphthotriazole exhibits a more red-shifted absorption and emission effect than benzotriazole. In summary, for photovoltaic devices, a light conversion agent with naphthotriazole as the core has a better light conversion performance. At the same time, naphthotriazole has more substitution sites, making it more conducive to adjusting and optimizing the performance of the light conversion agents. Therefore, when using naphthotriazole as the luminescent core, and some electron donating groups such as aryl, heteroaryl, and amino, etc., are used as auxochromic groups at the periphery of the naphthotriazole ring, it is easy to make the naphthotriazole molecule have high luminescence and strong absorption characteristics. Further, When the H atom on naphthotriazole is substituted with some alkyl, alkenyl, and ester group chains, especially long-chain substituents, it can not only effectively improve the solubility of the light conversion agent, especially the alkyl substituent can also form a layer of protective film at the periphery of the luminescent group, thereby improving the light stability of the light conversion adhesive film material. Meanwhile, the presence of such alkyl substituents can also avoid the luminescence quenching phenomenon caused by the stacking of fused ring aromatics, thereby improving the overall luminescence efficiency of the light conversion adhesive film material. Therefore, the light conversion adhesive film obtained by using the light conversion adhesive film composition of this disclosure has excellent light stability and high luminous efficiency, which can effectively function in the long term, thereby improving the service life of the photovoltaic devices (such as photovoltaic modules).

### DETAILED DESCRIPTION OF THE EMBODIMENTS

It should be noted that the embodiments and features in the embodiments in the present disclosure can be combined with each other without conflicting. The present disclosure will be described in detail below in combination with embodiments.

As analyzed in the background technology, the traditional light conversion adhesive film in the prior art has the problems of low photoelectric conversion efficiency, poor stability, and short service life. To solve such problems, the present disclosure provides a light conversion agent, a light conversion adhesive film composition, a light conversion adhesive film, and a photovoltaic module.

In a typical embodiment of this disclosure, a light conversion agent is provided, the light conversion agent is a naphthotriazole compound, and the naphthotriazole compound has the structural general formula as shown as follows:

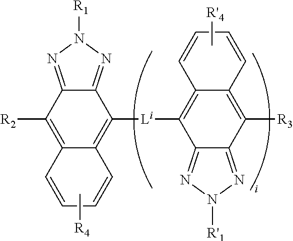

Wherein, i is any integer from 0 to 100; Lⁱ is independently selected from any one of a substituted or unsubstituted alkylene, a substituted or unsubstituted alkenylene, a substituted or unsubstituted arylene, and a substituted or unsubstituted heteroarylene; R₁ and R′₁ are each independently selected from any one of a substituted or unsubstituted C₁-C₂₀ alkyl, a substituted or unsubstituted C₂-C₂₀ heteroalkyl, a substituted or unsubstituted C₂-C₂₀ alkenyl, a substituted or unsubstituted C₆-C₄₀ aryl, a substituted or unsubstituted C₄-C₄₀ heteraryl, a substituted or unsubstituted C₂-C₂₀ ester group, a C₁-C₂₀ hydrocarbyl substituted or unsubstituted amino, a C₆-C₃₀ aryl substituted or unsubstituted amino, a C₁-C₂₀ hydrocarbyl substituted or unsubstituted amido, a substituted or unsubstituted C₃-C₂₀ cyclic amido, a substituted or unsubstituted C₃-C₂₀ cyclic imide group, a C₁-C₂₀ hydrocarbyl substituted or unsubstituted carboxyl, a C₁-C₂₀ hydrocarbylsubstituted or unsubstituted carbonyl, and a C₁-C₂₀ hydrocarbyl substituted or unsubstituted hydroxyl; R₂, R₃, R₄ and R′₄ are each independently selected from any one of H, a substituted or unsubstituted C₁-C₂₀ alkyl, a substituted or unsubstituted C₂-C₂₀ heteroalkyl, a substituted or unsubstituted C₂-C₂₀ alkenyl, a substituted or unsubstituted C₆-C₄₀ aryl, a substituted or unsubstituted C₄-C₄₀ heteraryl, a substituted or unsubstituted C₂-C₂₀ ester group, a C₁-C₂₀ hydrocarbyl substituted or unsubstituted amino, a C₆-C₃₀ aryl substituted or unsubstituted amino, a C₁-C₂₀ hydrocarbyl substituted or unsubstituted amido, a substituted or unsubstituted C₃-C₂₀ cyclic amido, a substituted or unsubstituted C₃-C₂₀ cyclic imide group, a C₁-C₂₀ hydrocarbyl substituted or unsubstituted carboxyl, a C₁-C₂₀ hydrocarbyl substituted or unsubstituted carbonyl, and a C₁-C₂₀ hydrocarbyl substituted or unsubstituted hydroxyl; wherein, the heteroatoms in heteroalkyl, heteroaryl, and heteroarylene groups are selected from any one or more of N, O, and S; and one or more methylene groups in the R₁, R′₁, R₂, R₃, R₄ and R′₄ are optionally substituted by —O— or —S—.

The triazole structure, as a building block of a classic optoelectronic functional material, has very unique electronic properties. Specifically, the three connected N atoms on the five-membered aromatic ring of the triazole structure not only enable the triazole to have both electron donating and electron withdrawing abilities, but also enable the triazole to have a higher electron cloud density compared to imidazole. When the triazole structure is connected with an aromatic ring to form a structure such as benzotriazole or naphthotriazole, the low-density π electron cloud of the benzene ring can effectively alleviate the electron cloud density on the triazole, so that the benzotriazole and naphthotriazole structures become very special electron-rich acceptors. Such electron-rich receptor structure can form an effective conjugated system even when it is connected with some electron donating groups having weaker electron donating abilities, thereby achieving electron transition and recovery from the ground state to the excited state, and radioluminescence. It should be specifically noted that compared to benzotriazole, the naphthotriazole with an additional benzene ring in this disclosure has a larger conjugate plane, which enables the naphthotriazole to have better coordination with respect to both electron donating and electron withdrawing. Therefore, when some auxochrome groups are attached to the periphery of the naphthotriazole ring, the naphthotriazole ring can often exhibit a more red-shifted absorption and emission effect. In addition, an additional benzene ring will inevitably lead to a lower LUMO for naphthotriazole compared to benzotriazole. Therefore, when the peripheral auxochrome groups of the naphthotriazole ring are identical, the naphthotriazole exhibits a more red-shifted absorption and emission effect than benzotriazole. In summary, for photovoltaic devices, a light conversion agent with naphthotriazole as the core has a better light conversion performance. At the same time, naphthotriazole has more substitution sites, making it more conducive to adjusting and optimizing the performance of the light conversion agents. Therefore, when using naphthotriazole as the luminescent core, and some electron donating groups such as aryl, heteroaryl, and amino, etc., are used as auxochromic groups at the periphery of the naphthotriazole ring, it is easy to make the naphthotriazole molecule have high luminescence and strong absorption characteristics. Further, When the H atom on naphthotriazole is substituted with some alkyl, alkenyl, and ester group chains, especially long-chain substituents, it can not only effectively improve the solubility of the light conversion agent, especially the alkyl substituent can also form a layer of protective film at the periphery of the luminescent group, thereby improving the light stability of the light conversion adhesive film material. Meanwhile, the presence of such alkyl substituents can also avoid the luminescence quenching phenomenon caused by the stacking of fused ring aromatics, thereby improving the overall luminescence efficiency of the light conversion adhesive film material. Therefore, the light conversion adhesive film obtained by using the light conversion adhesive film composition of this disclosure has excellent light stability and high luminous efficiency, which can effectively function in the long term, thereby improving the service life of the photovoltaic devices (such as photovoltaic modules).

In addition, preferably, Lⁱ is independently selected from any one of a substituted or unsubstituted C₁-C₁₀ alkylene, a substituted or unsubstituted C₂-C₁₀ alkenylene, a substituted or unsubstituted C₄-C₄₀ arylene, or a substituted or unsubstituted C₂-C₄₀ heteroarylene; further, in some embodiments, preferably, at least one of Lis is selected from 1,2-ethenylidene, 1,4-phenylene, 1,1′-biphenyl-4,4′-diyl, naphthalene-2,6-diyl, naphthalene-1,4-diyl, 9H-fluoren-2,7-diyl, perylene-3,9-diyl, perylene-3,10-diyl pyrene-1,6-diyl, 1H-pyrrole-2,5-diyl, furan-2,5-diyl, thiophene-2,5-diyl, thieno[3,2-b]thiophene-2,5-diyl, benzo[c]thiophene-1,3-diyl, dibenzo[b,d]thiophene-2,8-diyl, 9H-carbazole-3,6-diyl, 9H-carbazole-2,7-diyl, dibenzo[b,d]furan-2,8-diyl, 10H-phenothiazine-3,7-diyl or 10H-phenothiazine-2,8-diyl; each part of which is optionally substituted.

In one embodiment of this disclosure, when the above R₁, R₂, R₃ and R₄ carry a substituent, the substituent is selected from any one or more of C₁-C₆ alkyl, C₁-C₆ alkoxyl, C₂-C₆ alkenyl, C₂-C₆ alkynyl, geminally linked cycloalkyl, C₃-C₆ heterocycloalkyl, phenyl, anilino, naphthyl, biphenyl, halogen, hydroxyl, carboxyl, nitro, trifluoromethyl, trifluoromethoxy, cyano, amino, amido, and C₂-C₁₀ ester group; preferably, the substituent is selected from any one or more of methyl, ethyl, propyl, butyl, pentyl, hexyl, methoxy, ethoxy, propoxy, butoxy, pentyloxy, hexyloxy, ethenyl, propenyl, butenyl, pentenyl, hexenyl, ethynyl, propynyl, butynyl, pentynyl, hexynyl, cyclopropyloxy, cyclobutoxy, cyclopentoxy, cyclohexoxy, phenyl, anilino, naphthyl, biphenyl, halogen, hydroxyl, carboxyl, nitro, trifluoromethyl, trifluoromethoxy, cyano, amino, formamido, methyl formate group, ethyl formate group, ethyl propionate group, butyl propionate group, ethyl butyrate group, butyl butyrate group, methyl hexanoate group, methyl heptanoate group, and ethyl caprylate group.

Compared to the structure of a light conversion agent with larger molecular weight, the naphthotriazole light conversion agent with relatively smaller molecular weight in this disclosure not only exhibits absolute advantages in production difficulty and cost control, but also small molecular naphthotriazole light conversion agent can often exhibit better light stability and adjustability. This may be related to the relatively small conjugated system of naphthotriazole light conversion agent. Therefore, when the types of relatively small substituents described above are selected within a certain range, not only the production costs can be saved, but also the structure of the light conversion agent can be optimized more simply and directly, so that its performance can be improved.

In one embodiment of this disclosure, the above R₁ is selected from any one of a substituted or unsubstituted C₂-C₁₀ linear alkyl, a substituted or unsubstituted C₃-C₁₅ branched alkyl, a substituted or unsubstituted C₂-C₁₀ heteroalkyl, a substituted or unsubstituted C₂-C₁₀ alkenyl, a substituted or unsubstituted C₂-C₁₀ ester group, a C₁-C₁₀ hydrocarbyl substituted or unsubstituted amino, a C₆-C₂₀ aryl substituted or unsubstituted amino, a C₁-C₁₀ hydrocarbyl substituted or unsubstituted amido, a substituted or unsubstituted C₃-C₁₀ cyclic amido, a substituted or unsubstituted C₃-C₁₀ cyclic imide group, a C₁-C₁₀ hydrocarbyl substituted or unsubstituted carboxyl, a C₁-C₁₀ hydrocarbyl substituted or unsubstituted carbonyl, and a C₁-C₁₀ hydrocarbyl substituted or unsubstituted hydroxyl; R₂, R₃ and R₄ are each independently selected from any one of H, a substituted or unsubstituted C₂-C₁₀ linear alkyl, a substituted or unsubstituted C₃-C₁₅ branched alkyl, a substituted or unsubstituted C₂-C₁₀ heteroalkyl, a substituted or unsubstituted C₂-C₁₀ alkenyl, a substituted or unsubstituted C₆-C₃₀ aryl, a substituted or unsubstituted C₄-C₂₀ heteraryl, a substituted or unsubstituted C₂-C₁₀ ester group, a C₁-C₁₀ hydrocarbyl substituted or unsubstituted amino, a C₆-C₂₀ aryl substituted or unsubstituted amino, a C₁-C₁₀ hydrocarbyl substituted or unsubstituted amido, a substituted or unsubstituted C₃-C₁₀ cyclic amido, a substituted or unsubstituted C₃-C₁₀ cyclic imide group, a C₁-C₁₀ hydrocarbyl substituted or unsubstituted carboxyl, a C₁-C₁₀ hydrocarbyl substituted or unsubstituted carbonyl, and a C₁-C₁₀ hydrocarbyl substituted or unsubstituted hydroxyl; preferably, the R₁, the R₂, the R₃ and the R₄ are each independently selected from any one of the following substituted or unsubstituted substituents: n-hexyl, n-heptyl, n-octyl, n-nonyl, n-decyl, isopropyl, isobutyl, isopentyl, isohexyl, isoheptyl, isooctyl, isononyl, isodecyl, cyclopropyloxy, cyclobutoxy, cyclopentoxy, cyclohexyloxy, hexyloxy, heptyloxy, octyloxy, nonyloxy, decyloxy, ethenyl, propenyl, butenyl, pentenyl, hexenyl, heptenyl, octenyl, nonenyl, decenyl, allyl, phenyl, naphthyl, biphenyl, furyl, thienyl, indolyl, pyridyl, benzofuryl, benzothiophenyl, methyl formate group, ethyl formate group, ethyl propionate group, butyl propionate group, ethyl butyrate group, butyl butyrate group, methyl hexanoate group, methyl heptanoate group, ethyl caprylate group, amino, formamido, acetamido, propionamido, butyramido, pentanamido, cyclopropionamido, cyclobutyramido, cyclopropanoylimino, cyclobutanoylimino, carboxyl, or carbonyl.

Preferably, when the R₁, the R₂, the R₃ and the R₄ are selected from the above substituents, it is helpful to regulate and optimize the electronic structure of the luminescent core group, while supplementing the other performance requirements for the light conversion agent when it is applied. Specifically, the presence of an alkyl can not only reduce the probability of luminescence quenching phenomenon caused by the stacking of luminescent cores of plane as much as possible, but also can enhance the solubility of the light conversion agent in the light conversion adhesive film composition, thereby improving the compatibility of the light conversion agent in the adhesive film. At the same time, an alkyl can also serve as a protective layer to improve the stability of the light conversion agent. The function of an alkenyl is similar to that of the alkyl, however if an alkenyl is chosen as the terminal alkenyl, the terminal alkenyl can also undergo free radical crosslinking reaction with the matrix resin of the adhesive film, thereby making the light conversion agent present more stably in the adhesive film and thus reducing the migration rate of the auxiliary agent due to high temperature environment in practical use scenarios. Ester groups and other substituents such as amino, amido, and carbonyl, etc., carrying heteroatoms therein can not only function similarly to the alkyl, but also their heteroatoms, especially N, O, and S atoms, can also form non-covalent interactions with heteroatoms in the matrix resin of the adhesive film, such as a hydrogen bond, so that the movement of the entire light conversion agent molecule in the adhesive film is hindered, the rigidity is enhanced, and thus the energy loss is reduced, achieving the goal of improving light conversion efficiency. Moreover, substituents such as aryl and alkenyl, etc., can also play a role of regulating the electronic structure of the light conversion agent, so that the light conversion performance of the light conversion agent can be adjusted and optimized according to usage requirements.

In one embodiment of this disclosure, the above naphthotriazole compound has the structural general formula as shown as follows:

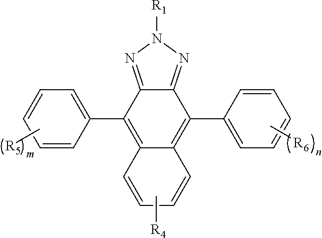

Wherein, m and n are each independently 1, 2, 3, or 4; R₁ is selected from any one of a substituted or unsubstituted C₁-C₁₂ linear alkyl, a substituted or unsubstituted C₃-C₁₀ branched alkyl, a substituted or unsubstituted C₂-C₁₆ alkenyl, a substituted or unsubstituted C₃-C₈ ester group, a C₅-C₈ hydrocarbyl substituted or unsubstituted amino, a C₆-C₁₀ aryl substituted or unsubstituted amino, a C₅-C₈ hydrocarbyl substituted or unsubstituted amido, a substituted or unsubstituted C₅-C₈ cyclic amido, a substituted or unsubstituted C₅-C₈ cyclic imide group, a C₅-C₈ hydrocarbyl substituted or unsubstituted carboxyl, a C₅-C₈ hydrocarbyl substituted or unsubstituted carbonyl, and a C₅-C₈ hydrocarbyl substituted or unsubstituted hydroxyl; preferably, R₄, R₅ and R₆ are each independently selected from any one of H, a substituted or unsubstituted C₁-C₁₂ linear alkyl, a substituted or unsubstituted C₃-C₁₀ branched alkyl, a substituted or unsubstituted C₂-C₁₆ alkenyl, a substituted or unsubstituted C₃-C₈ ester group, a C₅-C₈ hydrocarbyl substituted or unsubstituted amino, a C₆-C₁₀ aryl substituted or unsubstituted amino, a C₅-C₈ hydrocarbyl substituted or unsubstituted amido, a substituted or unsubstituted C₅-C₈ cyclic amido, a substituted or unsubstituted C₅-C₈ cyclic imide group, a C₅-C₈ hydrocarbyl substituted or unsubstituted carboxyl, a C₅-C₈ hydrocarbyl substituted or unsubstituted carbonyl, and a C₅-C₈ hydrocarbyl substituted or unsubstituted hydroxyl; preferably, the R₄ is H; preferably, the R₁, the R₅ and the R₆ are each independently selected from any one or more of a substituted or unsubstituted C₅-C₈ linear alkyl, a substituted or unsubstituted C₆-C₁₀ branched alkyl, a substituted or unsubstituted C₅-C₈ alkenyl, a substituted or unsubstituted C₃-C₈ ester group, and a phenyl substituted or unsubstituted amino, further, preferably, the R₁, the R₅ and the R₆ are each independent selected from any one of

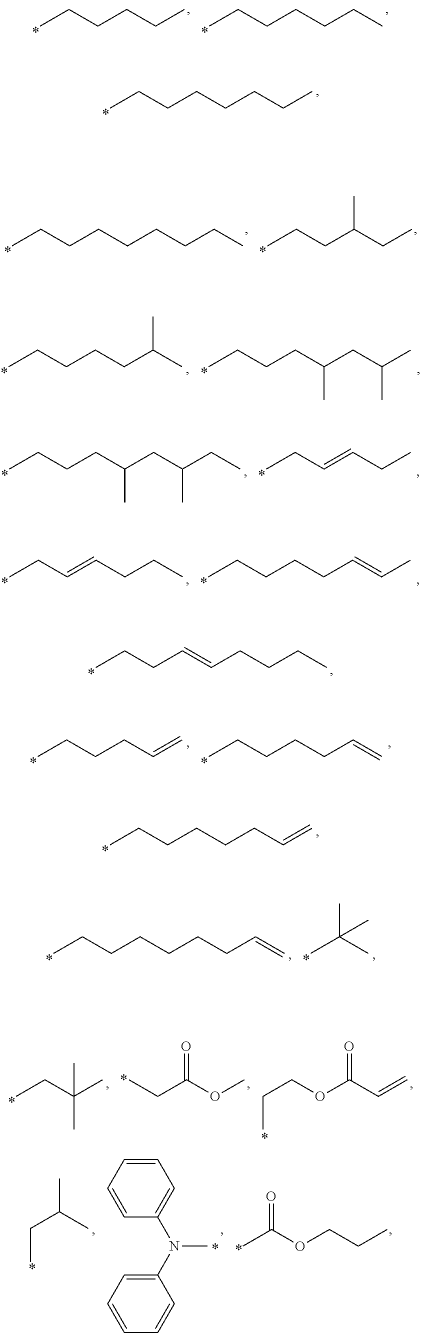

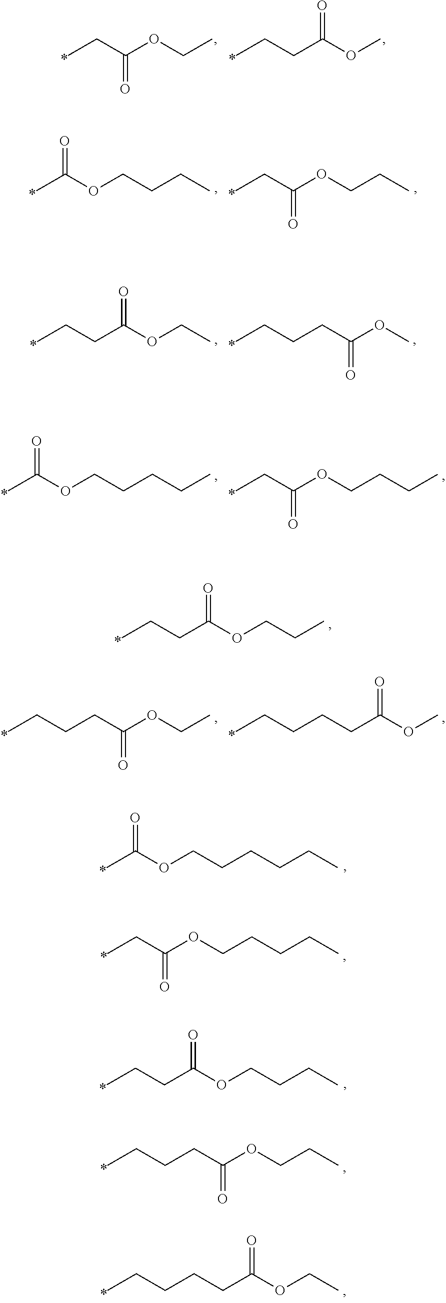

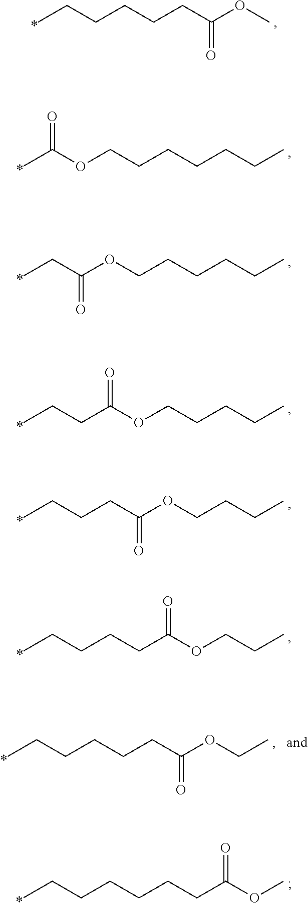

further, preferably, the R₁ is selected from any one of

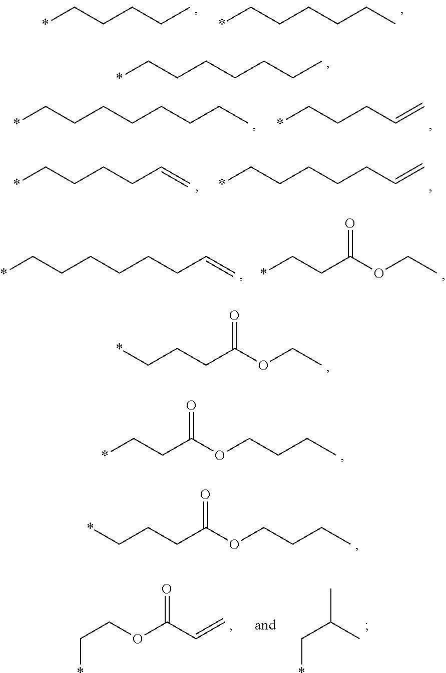

further, preferably, the R₁ is selected from any one of

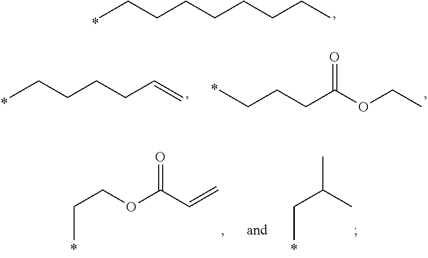

preferably, the R₅ and the R₆ are each independently selected from any one of

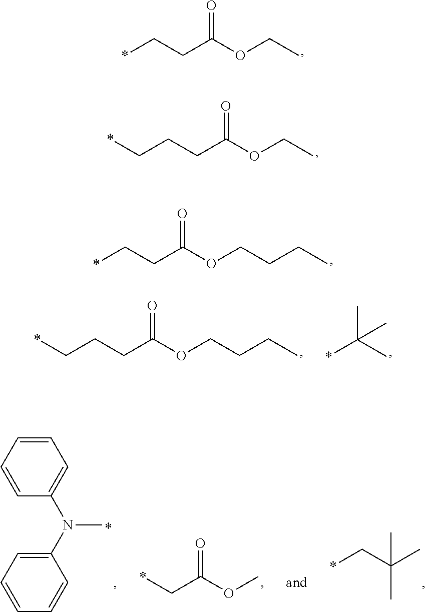

further, preferably, the R₅ and the R₆ are simultaneously any one of

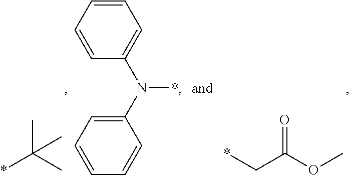

wherein, “*” indicates the attachment position of the substituent on the naphthotriazoles.

When further considering the performance requirements of a light conversion agent, it is appropriate to select a C₄-C₈ linear alkane, a C₄-C₈ alkene, a C₄-C₈ ester group, etc., for R₁, and select a short chain ester group and a tert-butyl group for R₅ and R₆. It is a better way for dividing the different substituents into different divisions to improve the overall performance of materials. If too long or too many substituted alkyl chains are present in the light conversion agent, there is a risk of insufficient rigidity for the light conversion agent, the light conversion agent may even become liquid, which will affect its light conversion efficiency. Considering the limitation of production cost on molecular weight, a C₄-C₈ substituent is a suitable choice on the whole. If there is no substituent on the benzene ring at the periphery of naphthotriazole, two adjacent molecules will inevitably undergo π-π stacking between the peripheral benzene rings, leading to luminescence quenching. Therefore, tert-butyl, as the smallest unit and a substituent with the best spatial effect, is obviously very suitable for use here on the whole.

In another typical embodiment of this disclosure, a light conversion adhesive film composition is provided, in terms of weight percentage, the light conversion adhesive film composition includes 80% to 99.98% of a matrix resin, 0.01% to 10% of a light conversion agent and 0.01% to 10% of an auxiliary agent, wherein the light conversion agent is the aforementioned light conversion agent.

Preferably, the content of the above light conversion adhesive film composition is conducive to fully utilizing the effects of each component and improving the overall synergistic coordination of each component. Preferably, the above mass ratio of light conversion agent to auxiliary agent is conducive to giving full play to the synergistic coordination of the two components, thereby enhancing the compatibility between the light conversion agent and the matrix resin. Thus, the absorption effect of naphthotriazole ring on broadband can be given full play. Furthermore, it will improve the overall luminous efficiency of the light conversion adhesive film material. Therefore, the light conversion adhesive film obtained by using the light conversion adhesive film composition of this disclosure has excellent light stability and high luminous efficiency, which can effectively function in the long term, thereby improving the service life of the photovoltaic devices (such as photovoltaic modules).

In one embodiment of this disclosure, in terms of weight percentage, the above light conversion adhesive film composition includes 98% to 99.98% of the matrix resin, 0.01% to 1% of the light conversion agent and 0.01% to 1% of the auxiliary agent; further, preferably, the mass ratio of the light conversion agent to the auxiliary agent is 1:2-500, preferably 1:2-50.

Preferably, the content of the components and the mass ratio of the light conversion agent to the auxiliary agent mentioned above are conducive to further improving the synergistic coordination of each component, thereby enhancing the crosslinking effect of the crosslinking agent and improving the performance of the light conversion adhesive film.

Preferably, the above matrix resin is selected from any one or more of EVA, PVA, PMMA, POE, and an organic silicon, in order to better coordinate with components such as light conversion agent, etc., and obtain high-performance light conversion adhesive film. The above matrix resins are inexpensive, which is conducive to reducing costs. Of course, those skilled in the art can also use other matrix resins, which will not be repeated here.

In one embodiment of this disclosure, the above auxiliary agent includes any one of a main crosslinking agent, an auxiliary crosslinking agent, a silane coupling agent, a light stabilizer and an inorganic powder, or a combination of at least two of the above.

Adding a crosslinking agent is conducive to improving the crosslinking rate of the cross-linked encapsulation adhesive film; adding a silane coupling agent is conducive to improving the interfacial interaction between the auxiliary crosslinking agent and the matrix resin, thereby enhancing the mechanical properties of the cross-linked encapsulation adhesive film. Further, preferably, the above auxiliary agents are conducive to improving their synergistic coordination with the light conversion agent, thereby enhancing the compatibility between the light conversion agent and the matrix resin. At the same time, it is conducive to increasing the overall crosslinking density of the light conversion agent, thereby improving the performance of the light conversion adhesive film.

In one embodiment of this disclosure, preferably, the crosslinking agent is selected from any one or more of tert-butyl peroxyisopropyl carbonate, 2,5-dimethyl2,5-bis(tert-butylperoxy)hexane, 1-bis(peroxytert-butyl)-3,3,5-trimethylcyclohexane), 2-ethylhexyl tert-butylperoxycarbonate, 2,5-dimethyl2,5-bis(tert-butylperoxy)hexane, 1,1-bis(tert-butylperoxy)-3,3,5-trimethylcyclohexane, 1,1-bis(tert-amylperoxy)-3,3,5-trimethylcyclohexane, 1,1-bis(tert-amylperoxy)cyclohexane, 2,2-bis(tert-butylperoxy)butane, tert-amyl peroxycarbonate, and tert-butyl peroxy-3,3,5-trimethylhexanoate.

In one embodiment of this disclosure, preferably, the auxiliary crosslinking agent is selected from any one or more of pentaerythritol triacrylate, pentaerythritol tetracrylate, ethoxylated pentaerythitol tetraacrylate, propoxylated pentaerythitol tetraacrylate, trimethylolpropane triacrylate, ethoxylated trimethylolpropane triacrylate, propoxylated trimethylolpropane triacrylate, ethoxylated glyceryl triacrylate, propoxylated glyceryl triacrylate, trimethylolpropane trimethacrylate, ethoxylated trimethylolpropane trimethacrylate, triethylene glycol dimethacrylate, diethylene glycol dimethacrylate, ethylene glycol dimethacrylate, polyethylene glycol(200) diacrylate, polyethylene glycol(400) diacrylate, polyethylene glycol(600) diacrylate, polyethylene glycol(200)dimethacrylate, polyethylene glycol(400)dimethacrylate, polyethylene glycol(600)dimethacrylate, ethoxylated bisphenol A diacrylate, ethoxylated bisphenol A dimethacrylate, 1,3-butylene glycol diacrylate, 1,4-butylene glycol diacrylate, and neopentyl glycol diacrylate.

In one embodiment of this disclosure, preferably, the silane coupling agent is selected from any one or more of vinyltrimethoxysilane, vinyltriethoxysilane, γ-methacryloxypropyltrimethoxysilane, 3-(2,3-epoxypropoxy) propyltrimethoxysilane, methacrylatesilane, or vinyltriisopropoxysilane.

In one embodiment of this disclosure, preferably, the light stabilizer is selected from any one or more of bis(2,2,6,6-tetramethyl-4-piperidinyl)sebacate, bis(1,2,2,6,6-pentamethyl-4-piperidinyl)sebacate, bis(1-octyloxy-2,2,6,6-tetramethyl-4-piperidinyl)sebacate, polysuccinic acid (4-hydroxy-2,2,6,6-tetramethyl-1-piperidylethanol)ester, 2-hydroxy-4-n-octyloxybenzophenone, 2-hydroxy-4-methoxybenzophenone, 2-hydroxy-4-methoxy-2′-carboxybenzophenone, and 2,4-dihydroxybenzophenone.

In one embodiment of this disclosure, preferably, the inorganic powder is selected from any one or more of magnesium hydroxide, magnesium oxide, or titanium dioxide, etc.

In yet another typical embodiment of this disclosure, a light conversion adhesive film is provided, which is obtained by mixing and molding an adhesive film composition, and the adhesive film composition is the aforementioned light conversion adhesive film composition.

In some preferred embodiments of this disclosure, the aforementioned light conversion adhesive film composition is uniformly mixed and then subjected to preparation processes such as melt extrusion molding, etc., at 80-120° C. to obtain a light conversion adhesive film. The resulting light conversion adhesive film has excellent stability and high luminous efficiency, and can effectively function in the long term, thereby improving the service life of the photovoltaic devices.

According to yet another embodiment of this disclosure, a photovoltaic module is provided, comprising a light conversion adhesive film, which is the aforementioned light conversion adhesive film.

The photovoltaic modules including the above-mentioned light conversion adhesive film of this disclosure have excellent photoelectric conversion efficiency. Of course, there are also more options according to different needs and application scenarios. The light conversion adhesive film of this disclosure is not limited to photovoltaic devices, agricultural films, building glass, and other fields.

The beneficial effects of this disclosure will be explained below in conjunction with specific embodiments and comparative embodiments.

### Embodiment 1

In terms of parts by weight, the light conversion adhesive film composition included 98.8 parts by weight of ethylene vinyl acetate, 0.2 parts by weight of naphthotriazole compound, 0.5 parts by weight of crosslinking agent tert-butyl peroxyisopropyl carbonate, 0.3 parts by weight of auxiliary crosslinking agent trimethylolpropane tetraacrylate, and 0.2 parts by weight of vinyltrimethoxy silane, the light conversion adhesive film composition was mixed evenly, then subjected to melt extrusion at 100° C. to form a film, so that the light conversion adhesive film was obtained.

Wherein, the naphthotriazole compound had the structural formula as shown as follows:

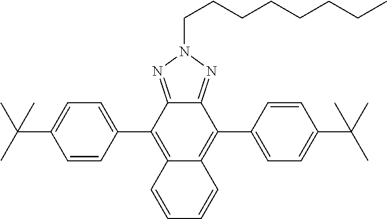 [SMILES: CCCCCCCCn1nc2c(-c3ccc(C(C)(C)C)cc3)c3ccccc3c(-c3ccc(C(C)(C)C)cc3)c2n1]

(marked as naphthotriazole compound a)

Further, the synthesis route of naphthotriazole compound was as shown as follows:

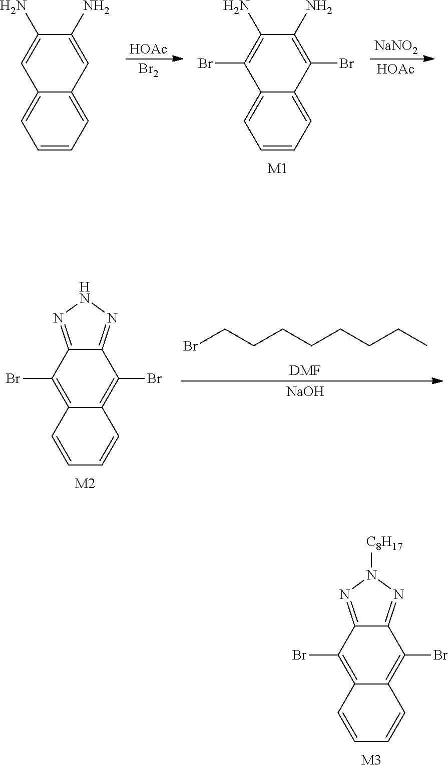

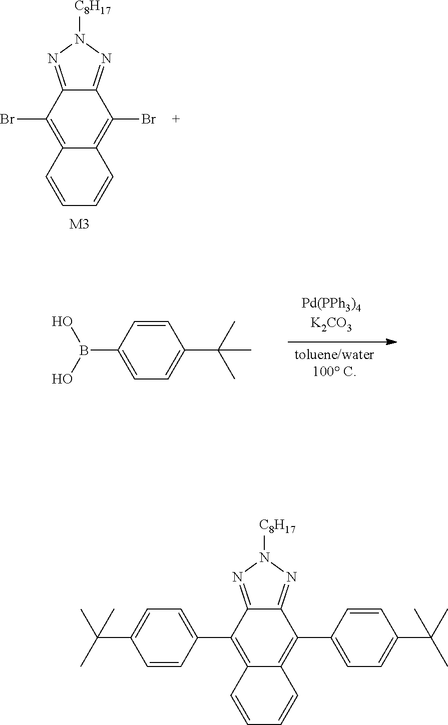

Specific steps were as follows:

### Synthesis of M1:

2,3-diaminonaphthalene (5 g, 31.6 mmol) was dissolved in 200 mL of acetic acid, then liquid bromine (15.2 g, 94.8 mmol) was added dropwise to the acetic acid solution under stirring. The reaction solution was stirred at room temperature for 3-6 hours, then the reaction was stopped. The reaction product was poured into water to precipitate, then filtered, and the filter cake was washed with a large amount of deionized water to obtain a off-white solid powder, which was the crude product. The crude product was purified by column chromatography method by using ethyl acetate/petroleum ether as the eluent, to finally obtain the pure M1 (7.5 g, 75%).

### Synthesis of M2:

M1 (5 g, 15.8 mmol) was dissolved in 100 mL of acetic acid, then sodium nitrite aqueous solution (2.2 g, 31.6 mmol) was added dropwise to the acetic acid solution under stirring to obtain a mixed solution. The mixed solution was stirred at room temperature for half an hour, then the product system was cooled down, filtered and washed with alkali to remove acid successively, to finally obtain a relatively pure white solid powder, which was M2 (4.65 g, 90%).

### Synthesis of M3:

M2 (4 g, 12.2 mmol) and sodium hydroxide (0.8 g) were dissolved in 80 mL of DMF to obtain a reaction system, then 1-bromo-n-octane (2.8 g, 14.6 mmol) was slowly added dropwise to the reaction system continuously under stirring. At 40° C., the reaction system was stirred overnight, then subjected to water washing, extraction, and separation successively to obtain the organic phase, the organic phase was dried over anhydrous magnesium sulfate and filtered to obtain the crude product, the crude product was mixed to obtain the mixed sample, which was separated and purified by column chromatography method by using a mixed solvent of ethyl acetate/petroleum ether as the eluent, and finally to obtain a relatively pure white solid powder, which was M3 (2.4 g, 45%).

### Synthesis of Naphthotriazole Compound a:

M3 (2 g, 4.6 mmol), 4-tertbutylphenylboronic acid (1.95 g, 10.9 mmol), potassium carbonate (2.54 g, 18.4 mmol), and tetrakis(triphenylphosphine)palladium (0.1 g) were mixed in a two-necked bottle to obtain a mixed system. The mixed system was displaced with nitrogen, then injected with 30 mL of toluene and 10 mL of deionized water. The mixed system was heated to 100° C., and stirred for 24 hours, and then the reaction was stopped to obtain the reaction product. The reaction product was subjected to water washing, extraction, and separation successively to obtain the organic phase, the organic phase was dried over anhydrous magnesium sulfate and filtered to obtain the crude product, the crude product was mixed to obtain the mixed sample, which was separated and purified by column chromatography method by using a mixed solvent of ethyl acetate/petroleum ether as the eluent, and finally to obtain a relatively pure beige white solid powder, which was the naphthotriazole compound a (2.06 g, 82%).

### Embodiment 2

The difference between this embodiment and Embodiment 1 lied in that the naphthotriazole compound had the structural formula as shown as follows:

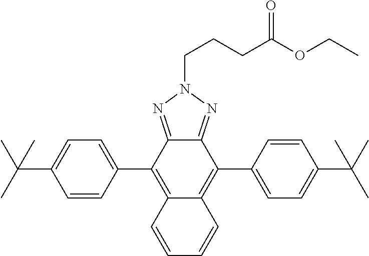 [SMILES: CCOC(=O)CCCn1nc2c(-c3ccc(C(C)(C)C)cc3)c3ccccc3c(-c3ccc(C(C)(C)C)cc3)c2n1]

(marked as naphthotriazole compound b), and finally the light conversion adhesive film was obtained.

Wherein, the synthesis route of naphthotriazole compound was as shown as follows:

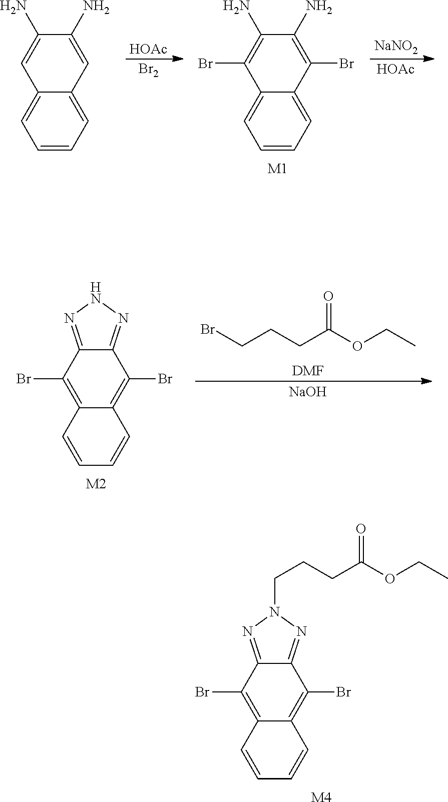

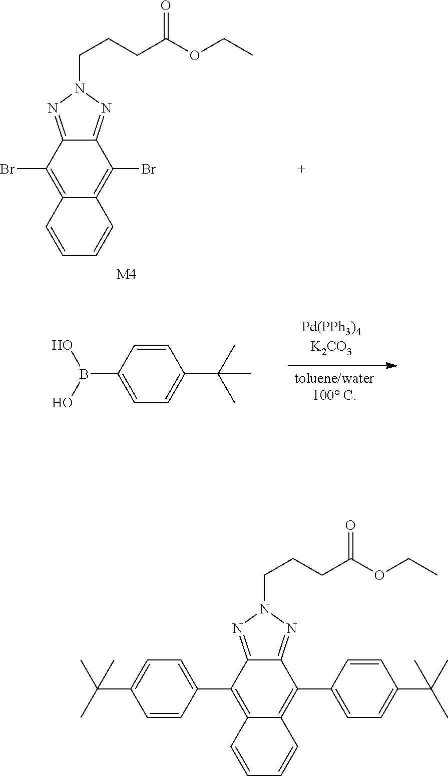

Specific steps were as follows:

The synthesis of M1 and M2 in Embodiment 2 was consistent with the synthesis process in Embodiment 1.

### Synthesis of M4:

M2 (4 g, 12.2 mmol) and sodium hydroxide (0.8 g) were dissolved in 80 mL of DMF to obtain a reaction system, then ethyl 4-bromobutyrate (2.86 g, 14.6 mmol) was slowly added dropwise to the reaction system under stirring. At 40° C., the reaction system was stirred overnight, then subjected to water washing, extraction, and separation successively to obtain the organic phase, the organic phase was dried over anhydrous magnesium sulfate and filtered to obtain the crude product, the crude product was mixed to obtain the mixed sample, which was separated and purified by column chromatography method by using a mixed solvent of ethyl acetate/petroleum ether as the eluent, and finally to obtain a relatively pure white solid powder, which was M4 (2.7 g, 50%).

### Synthesis of Naphthotriazole Compound b:

M4 (2 g, 4.5 mmol), 4-tertbutylphenylboronic acid (1.92 g, 10.8 mmol), potassium carbonate (2.48 g, 18 mmol), and tetrakis(triphenylphosphine)palladium (0.1 g) were mixed in a two-necked bottle to obtain a mixed system. The mixed system was displaced with nitrogen, then injected with 30 mL of toluene and 10 mL of deionized water. The mixed system was heated to 100° C., and stirred for 24 hours, and then the reaction was stopped. The reaction product was subjected to water washing, extraction, and separation successively to obtain the organic phase, the organic phase was dried over anhydrous magnesium sulfate and filtered to obtain the crude product, the crude product was mixed to obtain the mixed sample, which was separated and purified by column chromatography method by using a mixed solvent of ethyl acetate/petroleum ether as the eluent, and finally to obtain a relatively pure beige white solid powder, which was the naphthotriazole compound b (1.97 g, 80%).

### Embodiment 3

The difference between this embodiment and Embodiment 1 lied in that the naphthotriazole compound had the structural formula as shown as follows:

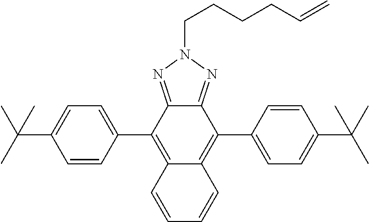 [SMILES: C=CCCCCn1nc2c(-c3ccc(C(C)(C)C)cc3)c3ccccc3c(-c3ccc(C(C)(C)C)cc3)c2n1]

(marked as naphthotriazole compound c), and finally the light conversion adhesive film was obtained.

Wherein, the synthesis route of naphthotriazole compound was as shown as follows:

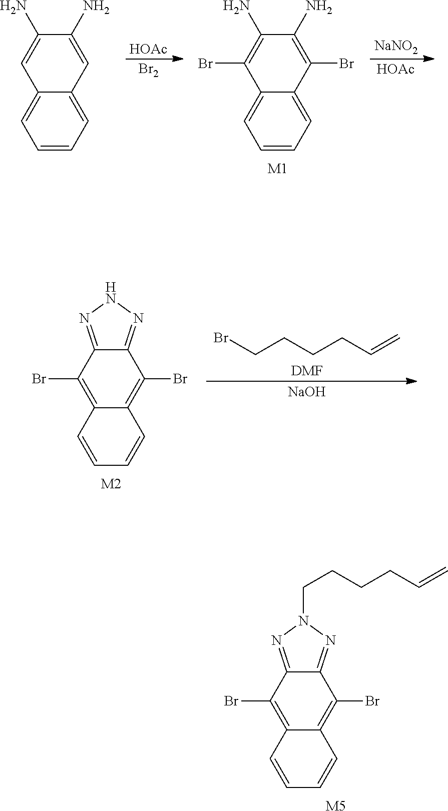

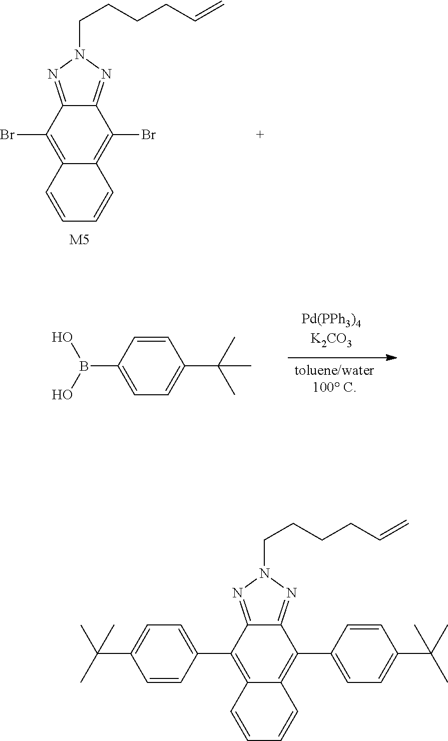

Specific steps were as follows:

The synthesis of M1 and M2 in Embodiment 3 was consistent with the synthesis process in Embodiment 1.

### Synthesis of M5:

M2 (4 g, 12.2 mmol) and sodium hydroxide (0.8 g) were dissolved in 80 mL of DMF to obtain a reaction system, then 6-bromo-1-hexene (2.38 g, 14.6 mmol) was slowly added dropwise to the reaction system under stirring. At 40° C., the reaction system was stirred overnight, then subjected to water washing, extraction, and separation successively to obtain the organic phase, the organic phase was dried over anhydrous magnesium sulfate and filtered to obtain the crude product, the crude product was mixed to obtain the mixed sample, which was separated and purified by column chromatography method by using a mixed solvent of ethyl acetate/petroleum ether as the eluent, and finally to obtain a relatively pure white solid powder, which was M5 (2.4 g, 48%).

### Synthesis of Naphthotriazole Compound c:

M5 (2 g, 4.9 mmol), 4-tertbutylphenylboronic acid (1.92 g, 10.8 mmol), potassium carbonate (2.7 g, 19.6 mmol), and tetrakis(triphenylphosphine)palladium (0.1 g) were mixed in a two-necked bottle to obtain a mixed system. The mixed system was displaced with nitrogen, then injected with 30 mL of toluene and 10 mL of deionized water. The mixed system was heated to 100° C., and stirred for 24 hours, and then the reaction was stopped to obtain the reaction product. The reaction product was subjected to water washing, extraction, and separation successively to obtain the organic phase, the organic phase was dried over anhydrous magnesium sulfate and filtered to obtain the crude product, the crude product was mixed to obtain the mixed sample, which was separated and purified by column chromatography method by using a mixed solvent of ethyl acetate/petroleum ether as the eluent, and finally to obtain a relatively pure beige white solid powder, which was the naphthotriazole compound c (2.15 g, 85%).

### Embodiment 4

The difference between this embodiment and Embodiment 1 lied in that the naphthotriazole compound had the structural formula as shown as follows:

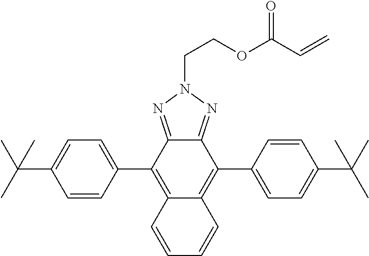 [SMILES: C=CC(=O)OCCn1nc2c(-c3ccc(C(C)(C)C)cc3)c3ccccc3c(-c3ccc(C(C)(C)C)cc3)c2n1]

(marked as naphthotriazole compound d), and finally the light conversion adhesive film was obtained.

Wherein, the synthesis route of naphthotriazole compound was as shown as follows:

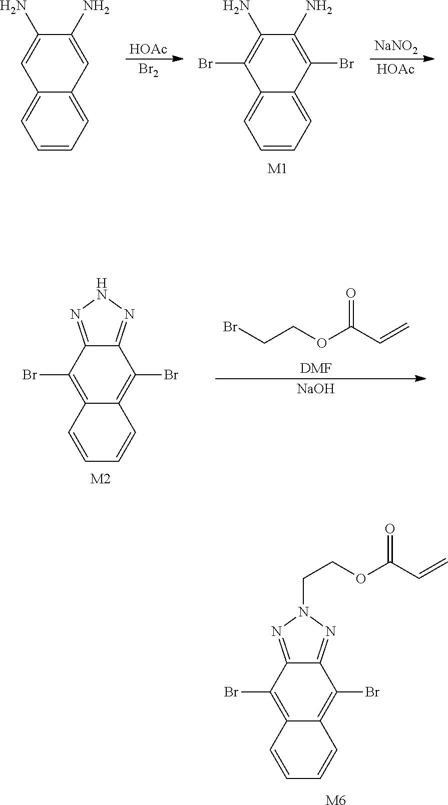

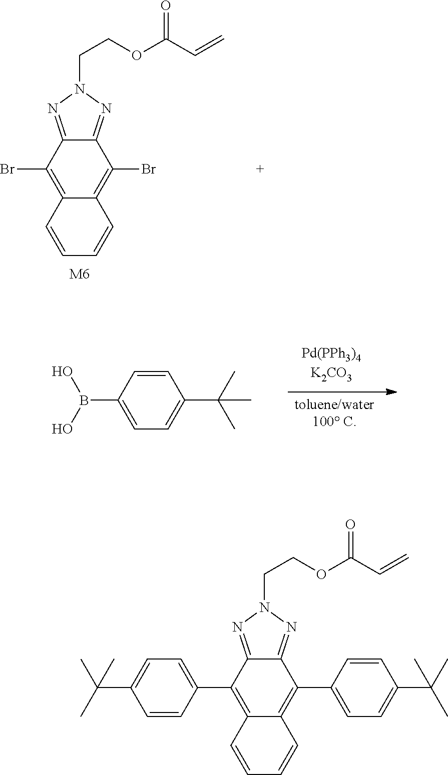

Specific steps were as follows:

The synthesis of M1 and M2 in Embodiment 4 was consistent with the synthesis process in Embodiment 1.

### Synthesis of M6:

M2 (4 g, 12.2 mmol) and sodium hydroxide (0.8 g) were dissolved in 80 mL of DMF to obtain a reaction system, then 2-bromoethyl acrylate (2.6 g, 14.6 mmol) was slowly added dropwise to the reaction system under stirring. At 40° C., the reaction system was stirred overnight, then subjected to water washing, extraction, and separation successively to obtain the organic phase, the organic phase was dried over anhydrous magnesium sulfate and filtered to obtain the crude product, the crude product was mixed to obtain the mixed sample, which was separated and purified by column chromatography method by using a mixed solvent of ethyl acetate/petroleum ether as the eluent, and finally to obtain a relatively pure white solid powder, which was M6 (2.18 g, 42%).

### Synthesis of Naphthotriazole Compound d:

M6 (2 g, 4.7 mmol), 4-tertbutylphenylboronic acid (1.84 g, 10.3 mmol), potassium carbonate (2.59 g, 18.8 mmol), and tetrakis(triphenylphosphine)palladium (0.1 g) were mixed in a two-necked bottle to obtain a mixed system. The mixed system was displaced with nitrogen, then injected with 30 mL of toluene and 10 mL of deionized water. The mixed system was heated to 100° C., and stirred for 24 hours, and then the reaction was stopped. The reaction product was subjected to water washing, extraction, and separation successively to obtain the organic phase, the organic phase was dried over anhydrous magnesium sulfate and filtered to obtain the crude product, the crude product was mixed to obtain the mixed sample, which was separated and purified by column chromatography method by using a mixed solvent of ethyl acetate/petroleum ether as the eluent, and finally to obtain a relatively pure beige white solid powder, which was the naphthotriazole compound d (2.05 g, 82%).

### Embodiment 5

The difference between this embodiment and Embodiment 1 lied in that the naphthotriazole compound had the structural formula as shown as follows:

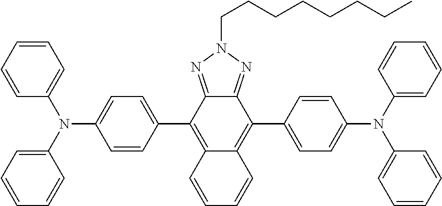 [SMILES: CCCCCCCCn1nc2c(-c3ccc(N(c4ccccc4)c4ccccc4)cc3)c3ccccc3c(-c3ccc(N(c4ccccc4)c4ccccc4)cc3)c2n1]

(marked as naphthotriazole compound e), and finally the light conversion adhesive film was obtained.

Wherein, the synthesis route of naphthotriazole compound was as shown as follows:

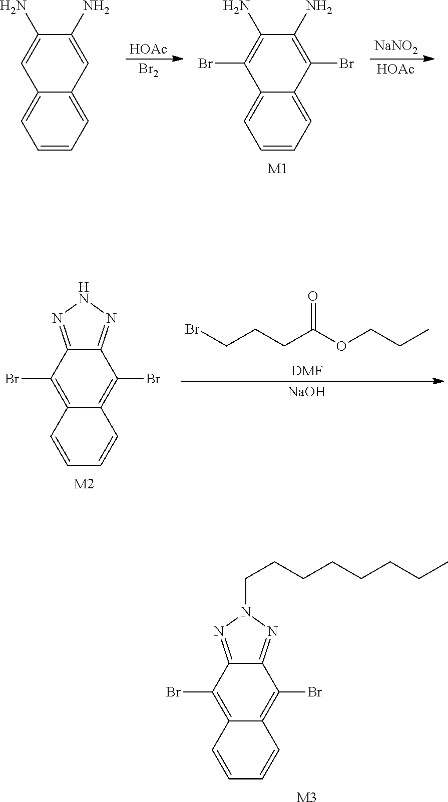

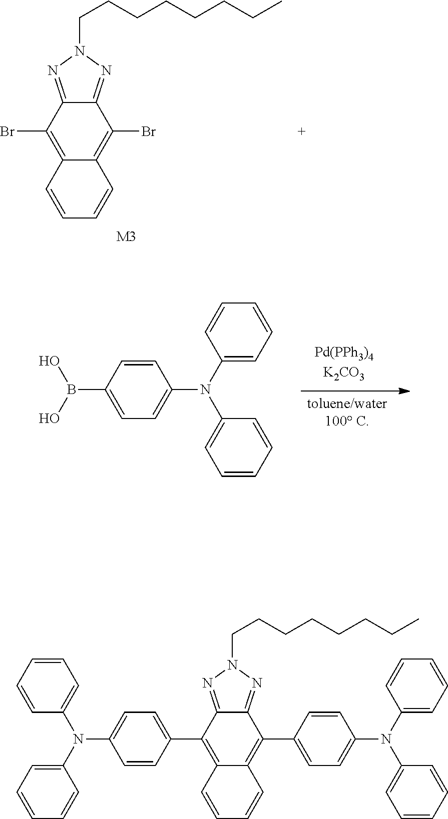

Specific steps were as follows:

The synthesis of M1, M2 and M3 in Embodiment 5 was consistent with the synthesis process in Embodiment 1.

### Synthesis of Naphthotriazole Compound e:

M3 (2 g, 4.6 mmol), 4-boronic acid triphenylamine (3.15 g, 10.9 mmol), potassium carbonate (2.54 g, 18.4 mmol), and tetrakis(triphenylphosphine)palladium (0.1 g) were mixed in a two-necked bottle to obtain a mixed system. The mixed system was displaced with nitrogen, then injected with 30 mL of toluene and 10 mL of deionized water. The mixed system was heated to 100° C., and stirred for 24 hours, and then the reaction was stopped. The reaction product was subjected to water washing, extraction, and separation successively to obtain the organic phase, the organic phase was dried over anhydrous magnesium sulfate and filtered to obtain the crude product, the crude product was mixed to obtain the mixed sample, which was separated and purified by column chromatography method by using a mixed solvent of ethyl acetate/petroleum ether as the eluent, and finally to obtain a relatively pure beige white solid powder, which was the naphthotriazole compound e (2.76 g, 78%).

### Embodiment 6

The difference between this embodiment and Embodiment 1 lied in that the naphthotriazole compound had the structural formula as shown as follows:

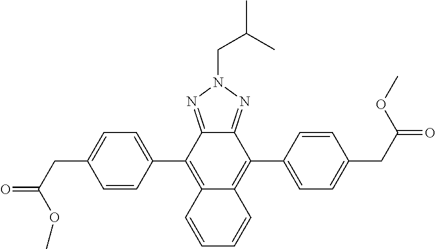 [SMILES: COC(=O)Cc1ccc(-c2c3ccccc3c(-c3ccc(CC(=O)OC)cc3)c3nn(CC(C)C)nc23)cc1]

(marked as naphthotriazole compound f), and finally the light conversion adhesive film was obtained.

Wherein, the synthesis route of naphthotriazole compound was as shown as follows:

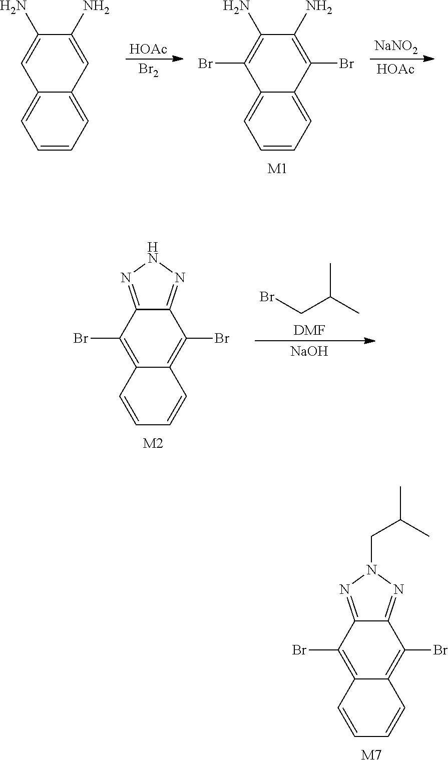

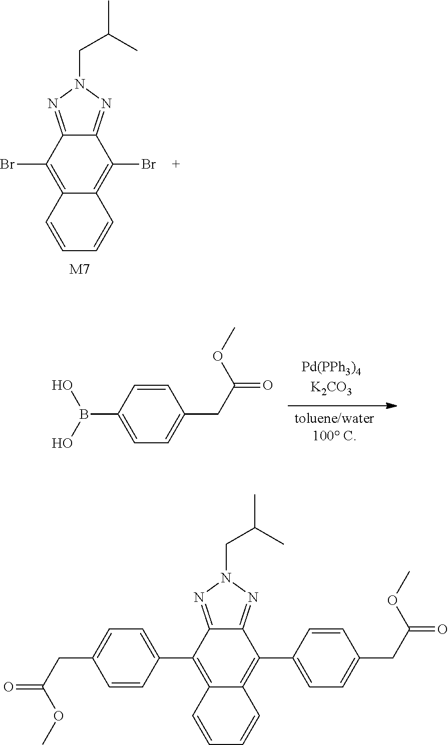

Specific steps were as follows:

The synthesis of M1 and M2 in Embodiment 6 was consistent with the synthesis process in Embodiment 1.

### Synthesis of M7:

M2 (4 g, 12.2 mmol) and sodium hydroxide (0.8 g) were dissolved in 80 mL of DMF to obtain a reaction system, then bromoisobutane (2.0 g, 14.6 mmol) was slowly added dropwise to the reaction system under stirring. At 40° C., the reaction system was stirred overnight, then subjected to water washing, extraction, and separation successively to obtain the organic phase, the organic phase was dried over anhydrous magnesium sulfate and filtered to obtain the crude product, the crude product was mixed to obtain the mixed sample, which was separated and purified by column chromatography method by using a mixed solvent of ethyl acetate/petroleum ether as the eluent, and finally to obtain a relatively pure white solid powder, which was M7 (2.48 g, 52%).

### Synthesis of Naphthotriazole Compound f:

M7 (2 g, 5.2 mmol), 4-(2-methoxy-2-oxyethyl)phenylboronic acid (2.22 g, 11.4 mmol), potassium carbonate (2.87 g, 20.8 mmol), and tetrakis(triphenylphosphine)palladium (0.1 g) were mixed in a two-necked bottle to obtain a mixed system. The mixed system was displaced with nitrogen, then injected with 30 mL of toluene and 10 mL of deionized water. The mixed system was heated to 100° C., and stirred for 24 hours, and then the reaction was stopped. The reaction product was subjected to water washing, extraction, and separation successively to obtain the organic phase, the organic phase was dried over anhydrous magnesium sulfate and filtered to obtain the crude product, the crude product was mixed to obtain the mixed sample, which was separated and purified by column chromatography method by using a mixed solvent of ethyl acetate/petroleum ether as the eluent, and finally to obtain a relatively pure beige white solid powder, which was the naphthotriazole compound f (2.17 g, 80%).

### Embodiment 7

The difference between this embodiment and Embodiment 1 lied in that, in terms of parts by weight, the light conversion adhesive film composition included 98 parts by weight of ethylene vinyl acetate, 1 part by weight of naphthotriazole compound a, 0.5 parts by weight of crosslinking agent tert-butyl peroxyisopropyl carbonate, 0.3 parts by weight of auxiliary crosslinking agent trimethylolpropane tetraacrylate, and 0.2 parts by weight of vinyltrimethoxy silane, and finally the light conversion adhesive film was obtained.

### Embodiment 8

The difference between this embodiment and Embodiment 1 lied in that, in terms of parts by weight, the light conversion adhesive film composition included 98 parts by weight of ethylene vinyl acetate, 0.67 part by weight of naphthotriazole compound a, 0.63 parts by weight of crosslinking agent tert-butyl peroxyisopropyl carbonate, 0.4 parts by weight of auxiliary crosslinking agent trimethylolpropane tetraacrylate, and 0.3 parts by weight of vinyltrimethoxy silane, and finally the light conversion adhesive film was obtained.

### Embodiment 9

The difference between this embodiment and Embodiment 1 lied in that, in terms of parts by weight, the light conversion adhesive film composition included 98 parts by weight of ethylene vinyl acetate, 0.039 part by weight of naphthotriazole compound a, 1 part by weight of crosslinking agent tert-butyl peroxyisopropyl carbonate, 0.66 parts by weight of auxiliary crosslinking agent trimethylolpropane tetraacrylate, and 0.301 parts by weight of vinyltrimethoxy silane, and finally the light conversion adhesive film was obtained.

### Embodiment 10

The difference between this embodiment and Embodiment 1 lied in that, in terms of parts by weight, the light conversion adhesive film composition included 98 parts by weight of ethylene vinyl acetate, 0.08 part by weight of naphthotriazole compound a, 1 part by weight of crosslinking agent tert-butyl peroxyisopropyl carbonate, 0.62 parts by weight of auxiliary crosslinking agent trimethylolpropane tetraacrylate, and 0.3 parts by weight of vinyltrimethoxy silane, and finally the light conversion adhesive film was obtained.

### Embodiment 11

The difference between this embodiment and Embodiment 1 lied in that, in terms of parts by weight, the matrix resin was ethylene-1-octene, and finally the light conversion adhesive film was obtained.

### Comparative Embodiment 1

The difference between this embodiment and Embodiment 1 lied in that, the light conversion agent was

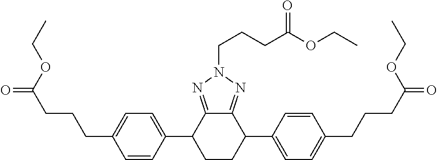 [SMILES: CCOC(=O)CCCc1ccc(C2CCC(c3ccc(CCCC(=O)OCC)cc3)c3nn(CCCC(=O)OCC)nc32)cc1]

(which was published in the Chinese patent disclosure document with disclosure number 202310353493.6), and finally the light conversion adhesive film was obtained.

### Comparative Embodiment 2

The difference between this embodiment and Embodiment 1 lied in that, the light conversion agent was

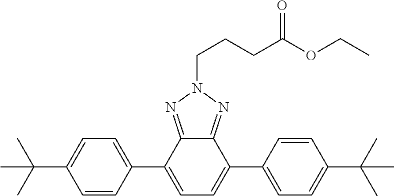 [SMILES: CCOC(=O)CCCn1nc2c(-c3ccc(C(C)(C)C)cc3)ccc(-c3ccc(C(C)(C)C)cc3)c2n1]

(which was published in the Chinese patent disclosure document with disclosure number 202310353493.6), and finally the light conversion adhesive film was obtained.

### Comparative Embodiment 3

The difference between this embodiment and Embodiment 1 lied in that, the light conversion agent was

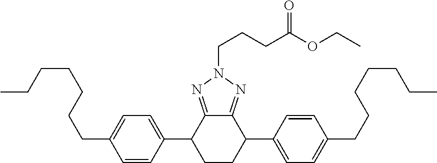 [SMILES: CCCCCCCc1ccc(C2CCC(c3ccc(CCCCCCC)cc3)c3nn(CCCC(=O)OCC)nc32)cc1]

(which was published in the Chinese patent disclosure document with disclosure number 202310353493.6), and finally the light conversion adhesive film was obtained.

### Comparative Embodiment 4

The difference between this embodiment and Embodiment 1 lied in that, the light conversion agent was

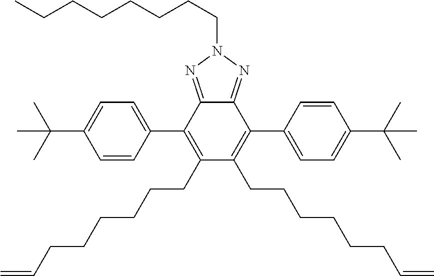 [SMILES: C=CCCCCCCc1c(CCCCCCC=C)c(-c2ccc(C(C)(C)C)cc2)c2nn(CCCCCCCC)nc2c1-c1ccc(C(C)(C)C)cc1]

(which was published in the Chinese patent disclosure document with disclosure number 202311535255.3), and finally the light conversion adhesive film was obtained.

### Comparative Embodiment 5

The difference between this embodiment and Embodiment 1 lied in that, the light conversion agent was

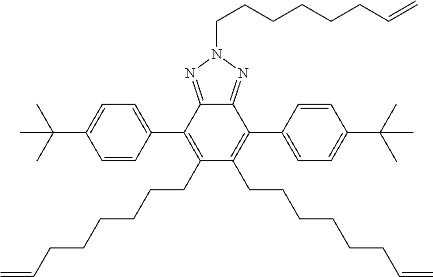 [SMILES: C=CCCCCCCc1c(CCCCCCC=C)c(-c2ccc(C(C)(C)C)cc2)c2nn(CCCCCCC=C)nc2c1-c1ccc(C(C)(C)C)cc1]

(which was published in the Chinese patent disclosure document with disclosure number 202311535255.3), and finally the light conversion adhesive film was obtained.

### Comparative Embodiment 6

The difference between this embodiment and Embodiment 1 lied in that, the light conversion agent was

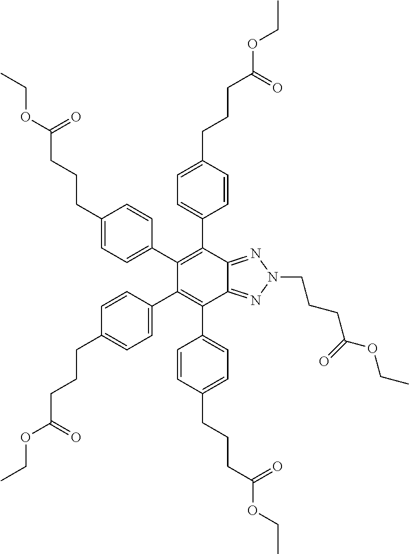 [SMILES: CCOC(=O)CCCc1ccc(-c2c(-c3ccc(CCCC(=O)OCC)cc3)c(-c3ccc(CCCC(=O)OCC)cc3)c3nn(CCCC(=O)OCC)nc3c2-c2ccc(CCCC(=O)OCC)cc2)cc1]

(which was published in the Chinese patent disclosure document with disclosure number 202311712843.X), and finally the light conversion adhesive film was obtained.

### Comparative Embodiment 7

The difference between this embodiment and Embodiment 1 lied in that, the light conversion agent was

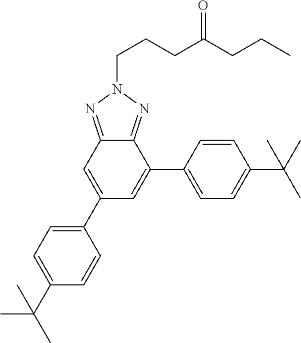 [SMILES: CCCC(=O)CCCn1nc2cc(-c3ccc(C(C)(C)C)cc3)cc(-c3ccc(C(C)(C)C)cc3)c2n1]

(which was published in the Chinese patent disclosure document with disclosure number 202311712843.X), and finally the light conversion adhesive film was obtained.

### Comparative Embodiment 8

The difference between this embodiment and Embodiment 1 lied in that, in terms of parts by weight, the light conversion adhesive film composition included 70 parts by weight of matrix resin, 15 part by weight of naphthotriazole compound a, 5 parts by weight of crosslinking agent tert-butyl peroxyisopropyl carbonate, 5 parts by weight of auxiliary crosslinking agent trimethylolpropane tetraacrylate, and 5 parts by weight of vinyltrimethoxy silane, and finally the light conversion adhesive film was obtained.

### Test Methods

The light conversion adhesive films obtained in the above embodiments and comparative embodiments of this disclosure were made into photovoltaic modules, specifically including stacking the glass, the light conversion adhesive film and the glass sequentially, and then vacuum laminating to obtain the pre-pressed modules, and the following performances of the obtained photovoltaic modules were tested by the testing methods below, and the above test results are listed in Table 1.

Light conversion efficiency: Horiba spectrometer FL-3 was used for the absolute quantum efficiency test at room temperature using an integrating sphere.

Aging test: the upper and lower surfaces of the light conversion adhesive films obtained in the above embodiments and comparative embodiments were stacked with glass layers, respectively to obtain the pre-pressed modules, which were subjected to UV300 aging test in a multiple UV aging chamber (power of 142 W, temperature of 70° C.).

Yellowing index: the yellowing indexes (ΔYI) of the pre-pressed modules before and after the aging testing were determined in accordance with the national standard GB 2409 “Plastics-Test Method of Yellowness Index”.

Water vapor transmittance: The test method was made reference to the standard GB/T 29848 “Ethylene-Vinyl Acetate Copolymer (EVA) Adhesive Film for Photovoltaic Module Encapsulation”.

TABLE 1

| | | | | Stability |
|---|---|---|---|---|
| | | | Light | (yellowing |
| Embodiments/Comparative | Absorption | Emission | conversion | index |
| embodiments | wavelength | wavelength | efficiency/% | ΔYI) |
| Embodiment 1 | 395 nm | 495 nm | 88% | 0.55 |
| Embodiment 2 | 395 nm | 496 nm | 90% | 0.48 |
| Embodiment 3 | 394 nm | 496 nm | 88% | 0.52 |
| Embodiment 4 | 400 nm | 505 nm | 85% | 0.46 |
| Embodiment 5 | 375 nm | 478 nm | 92% | 0.60 |
| Embodiment 6 | 397 nm | 500 nm | 88% | 0.50 |
| Embodiment 7 | 395 nm | 495 nm | 82% | 0.75 |
| Embodiment 8 | 395 nm | 495 nm | 87% | 0.60 |
| Embodiment 9 | 395 nm | 495 nm | 72% | 0.35 |
| Embodiment 10 | 395 nm | 495 nm | 82% | 0.45 |
| Embodiment 11 | 395 nm | 495 nm | 85% | 0.52 |
| Comparative embodiment 1 | 350 nm | 425 nm | 87% | 0.54 |
| Comparative embodiment 2 | 348 nm | 422 nm | 82% | 0.50 |
| Comparative embodiment 3 | 347 nm | 420 nm | 83% | 0.48 |
| Comparative embodiment 4 | 355 nm | 430 nm | 75% | 0.45 |
| Comparative embodiment 5 | 355 nm | 430 nm | 77% | 0.43 |
| Comparative embodiment 6 | 330 nm | 410 nm | 75% | 0.52 |
| Comparative embodiment 7 | 343 nm | 430 nm | 75% | 0.55 |
| Comparative embodiment 8 | 395 nm | 495 nm | 85% | 1.35 |

It could be seen from the above that compared with Comparative embodiments, both absorption wavelength and emission wavelength of the light conversion adhesive film obtained in the embodiments of this disclosure had significantly increased, indicating a significant redshift, the emission wavelength in the embodiments of this disclosure was closer to 500 nm, therefore the gain effect on the photovoltaic modules (such as the power of the photovoltaic modules) was better.

It could be seen from the above description that the above embodiments of the present disclosure have achieved the following technical effects:

The triazole structure, as a building block of a classic optoelectronic functional material, has very unique electronic properties. Specifically, the three connected N atoms on the five-membered aromatic ring of the triazole structure not only enable the triazole to have both electron donating and electron withdrawing abilities, but also enable the triazole to have a higher electron cloud density compared to imidazole. When the triazole structure is connected with an aromatic ring to form a structure such as benzotriazole or naphthotriazole, the low-density π electron cloud of the benzene ring can effectively alleviate the electron cloud density on the triazole, so that the benzotriazole and naphthotriazole structures become very special electron-rich acceptors. Such electron-rich receptor structure can form an effective conjugated system even when it is connected with some electron donating groups having weaker electron donating abilities, thereby achieving electron transition and recovery from the ground state to the excited state, and radioluminescence. It should be specifically noted that compared to benzotriazole, the naphthotriazole with an additional benzene ring in this disclosure has a larger conjugate plane, which enables the naphthotriazole to have better coordination with respect to both electron donating and electron withdrawing. Therefore, when some auxochrome groups are attached to the periphery of the naphthotriazole ring, the naphthotriazole ring can often exhibit a more red-shifted absorption and emission effect. In addition, an additional benzene ring will inevitably lead to a lower LUMO for naphthotriazole compared to benzotriazole. Therefore, when the peripheral auxochrome groups of the naphthotriazole ring are identical, the naphthotriazole exhibits a more red-shifted absorption and emission effect than benzotriazole. In summary, for photovoltaic devices, a light conversion agent with naphthotriazole as the core has a better light conversion performance. At the same time, naphthotriazole has more substitution sites, making it more conducive to adjusting and optimizing the performance of the light conversion agents. Therefore, when using naphthotriazole as the luminescent core, and some electron donating groups such as aryl, heteroaryl, and amino, etc., are used as auxochromic groups at the periphery of the naphthotriazole ring, it is easy to make the naphthotriazole molecule have high luminescence and strong absorption characteristics. Further, When the H atom on naphthotriazole is substituted with some alkyl, alkenyl, and ester group chains, especially long-chain substituents, it can not only effectively improve the solubility of the light conversion agent, especially the alkyl substituent can also form a layer of protective film at the periphery of the luminescent group, thereby improving the light stability of the light conversion adhesive film material. Meanwhile, the presence of such alkyl substituents can also avoid the luminescence quenching phenomenon caused by the stacking of fused ring aromatics, thereby improving the overall luminescence efficiency of the light conversion adhesive film material. Therefore, the light conversion adhesive film obtained by using the light conversion adhesive film composition of this disclosure has excellent light stability and high luminous efficiency, which can effectively function in the long term, thereby improving the service life of the photovoltaic devices (such as photovoltaic modules).

The above contents only describe the preferred embodiments of the present disclosure, and are not intended to limit the present disclosure. For those skilled in the art, various modifications and changes can be made to the present disclosure. Any modifications, equivalent substitutions, improvements, and the like made within the spirit and principle of the present disclosure shall be included within the scope of protection of the present disclosure.

What is claimed is:

1. A light conversion agent, wherein, the light conversion agent is a naphthotriazole compound, and the naphthotriazole compound has the structural general formula as shown as follows: 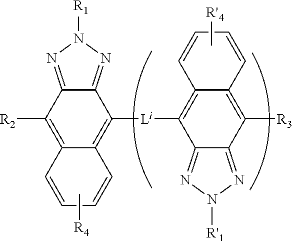
wherein, i is any integer from 0 to 100;
Lⁱ is independently selected from any one of a substituted or unsubstituted alkylene, a substituted or unsubstituted alkenylene, a substituted or unsubstituted arylene, or a substituted or unsubstituted heteroarylene;
R₁ and R′₁ are each independently selected from any one of a substituted or unsubstituted C₁-C₂₀ alkyl, a substituted or unsubstituted C₂-C₂₀ heteroalkyl, a substituted or unsubstituted C₂-C₂₀ alkenyl, a substituted or unsubstituted C₆-C₄₀ aryl, a substituted or unsubstituted C₄-C₄₀ heteraryl, a substituted or unsubstituted C₂-C₂₀ ester group, a C₁-C₂₀ hydrocarbyl substituted or unsubstituted amino, a C₆-C₃₀ aryl substituted or unsubstituted amino, a C₁-C₂₀ hydrocarbyl substituted or unsubstituted amido, a substituted or unsubstituted C₃-C₂₀ cyclic amido, a substituted or unsubstituted C₃-C₂₀ cyclic imide group, a C₁-C₂₀ hydrocarbyl substituted or unsubstituted carboxyl, a C₁-C₂₀ hydrocarbyl substituted or unsubstituted carbonyl, and a C₁-C₂₀ hydrocarbyl substituted or unsubstituted hydroxyl;
R₂, R₃, R₄ and R′₄ are each independently selected from any one of H, a substituted or unsubstituted C₁-C₂₀ alkyl, a substituted or unsubstituted C₂-C₂₀ heteroalkyl, a substituted or unsubstituted C₂-C₂₀ alkenyl, a substituted or unsubstituted C₆-C₄₀ aryl, a substituted or unsubstituted C₄-C₄₀ heteraryl, a substituted or unsubstituted C₂-C₂₀ ester group, a C₁-C₂₀ hydrocarbyl substituted or unsubstituted amino, a C₆-C₃₀ aryl substituted or unsubstituted amino, a C₁-C₂₀ hydrocarbyl substituted or unsubstituted amido, a substituted or unsubstituted C₃-C₂₀ cyclic amido, a substituted or unsubstituted C₃-C₂₀ cyclic imide group, a C₁-C₂₀ hydrocarbyl substituted or unsubstituted carboxyl, a C₁-C₂₀ hydrocarbyl substituted or unsubstituted carbonyl, and a C₁-C₂₀ hydrocarbyl substituted or unsubstituted hydroxyl;
wherein, the heteroatoms in heteroalkyl, heteroaryl, and heteroarylene groups are selected from any one or more of N, O, and S; and
one or more methylene groups in the R₁, the R′₁, the R₂, the R₃, the R₄ and the R′₄ are optionally substituted by —O— or —S—.

2. The light conversion agent according to claim 1, wherein, when the R₁, the R₂, the R₃ and the R₄ carry a substituent, the substituent is selected from any one or more of C₁-C₆ alkyl, C₁-C₆ alkoxyl, C₂-C₆ alkenyl, C₂-C₆ alkynyl, geminally linked cycloalkyl, C₃-C₆ heterocycloalkyl, phenyl, anilino, naphthyl, biphenyl, halogen, hydroxyl, carboxyl, nitro, trifluoromethyl, trifluoromethoxy, cyano, amino, amido, and C₂-C₁₀ ester group;
preferably, the substituent is selected from any one or more of methyl, ethyl, propyl, butyl, pentyl, hexyl, methoxy, ethoxy, propoxy, butoxy, pentyloxy, hexyloxy, ethenyl, propenyl, butenyl, pentenyl, hexenyl, ethynyl, propynyl, butynyl, pentynyl, hexynyl, cyclopropyloxy, cyclobutoxy, cyclopentoxy, cyclohexoxy, phenyl, anilino, naphthyl, biphenyl, halogen, hydroxyl, carboxyl, nitro, trifluoromethyl, trifluoromethoxy, cyano, amino, formamido, methyl formate group, ethyl formate group, ethyl propionate group, butyl propionate group, ethyl butyrate group, butyl butyrate group, methyl hexanoate group, methyl heptanoate group, and ethyl caprylate group.

3. The light conversion agent according to claim 1, wherein, the R₁ is selected from any one of a substituted or unsubstituted C₂-C₁₀ linear alkyl, a substituted or unsubstituted C₃-C₁₅ branched alkyl, a substituted or unsubstituted C₂-C₁₀ heteroalkyl, a substituted or unsubstituted C₂-C₁₀ alkenyl, a substituted or unsubstituted C₂-C₁₀ ester group, a C₁-C₁₀ hydrocarbyl substituted or unsubstituted amino, a C₆-C₂₀ aryl substituted or unsubstituted amino, a C₁-C₁₀ hydrocarbylsubstituted or unsubstituted amido, a substituted or unsubstituted C₃-C₁₀ cyclic amido, a substituted or unsubstituted C₃-C₁₀ cyclic imide group, a C₁-C₁₀ hydrocarbyl substituted or unsubstituted carboxyl, a C₁-C₁₀ hydrocarbyl substituted or unsubstituted carbonyl, and a C₁-C₁₀ hydrocarbyl substituted or unsubstituted hydroxyl; and
the R₂, the R₃ and the R₄ are each independently selected from any one of H, a substituted or unsubstituted C₂-C₁₀ linear alkyl, a substituted or unsubstituted C₃-C₁₅ branched alkyl, a substituted or unsubstituted C₂-C₁₀ heteroalkyl, a substituted or unsubstituted C₂-C₁₀ alkenyl, a substituted or unsubstituted C₆-C₃₀ aryl, a substituted or unsubstituted C₄-C₂₀ heteraryl, a substituted or unsubstituted C₂-C₁₀ ester group, a C₁-C₁₀ hydrocarbyl substituted or unsubstituted amino, a C₆-C₂₀ aryl substituted or unsubstituted amino, a C₁-C₁₀ hydrocarbyl substituted or unsubstituted amido, a substituted or unsubstituted C₃-C₁₀ cyclic amido, a substituted or unsubstituted C₃-C₁₀ cyclic imide group, a C₁-C₁₀ hydrocarbyl substituted or unsubstituted carboxyl, a C₁-C₁₀ hydrocarbyl substituted or unsubstituted carbonyl, and a C₁-C₁₀ hydrocarbyl substituted or unsubstituted hydroxyl.

4. The light conversion agent according to claim 1, wherein, the naphthotriazole compound has the structural general formula as shown as follows: 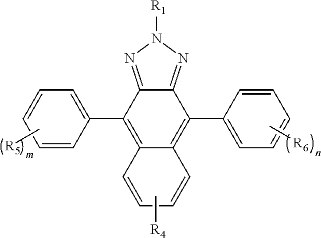
wherein, m and n are each independently 1, 2, 3, or 4;
the R₁ is selected from any one of a substituted or unsubstituted C₁-C₁₂ linear alkyl, a substituted or unsubstituted C₃-C₁₀ branched alkyl, a substituted or unsubstituted C₂-C₁₆ alkenyl, a substituted or unsubstituted C₃-C₈ ester group, a C₅-C₈ hydrocarbyl substituted or unsubstituted amino, a C₆-C₁₀ aryl substituted or unsubstituted amino, a C₅-C₈ hydrocarbyl substituted or unsubstituted amido, a substituted or unsubstituted C₅-C₈ cyclic amido, a substituted or unsubstituted C₅-C₈ cyclic imide group, a C₅-C₈ hydrocarbyl substituted or unsubstituted carboxyl, a C₅-C₈ hydrocarbyl substituted or unsubstituted carbonyl, and a C₅-C₈ hydrocarbyl substituted or unsubstituted hydroxyl;
Preferably, the R₄, R₅ and R₆ are each independently selected from any one of H, a substituted or unsubstituted C₁-C₁₂ linear alkyl, a substituted or unsubstituted C₃-C₁₀ branched alkyl, a substituted or unsubstituted C₂-C₁₆ alkenyl, a substituted or unsubstituted C₃-C₈ ester group, a C₅-C₈ hydrocarbyl substituted or unsubstituted amino, a C₆-C₁₀ aryl substituted or unsubstituted amino, a C₅-C₈ hydrocarbyl substituted or unsubstituted amido, a substituted or unsubstituted C₅-C₈ cyclic amido, a substituted or unsubstituted C₅-C₈ cyclic imide group, a C₅-C₈ hydrocarbyl substituted or unsubstituted carboxyl, a C₅-C₈ hydrocarbyl substituted or unsubstituted carbonyl, and a C₅-C₈ hydrocarbyl substituted or unsubstituted hydroxyl; preferably, the R₄ is H.

5. A light conversion adhesive film composition, wherein, in terms of weight percentage, the light conversion adhesive film composition comprises:
80% to 99.98% of a matrix resin;
0.01% to 10% of a light conversion agent; and
0.01% to 10% of an auxiliary agent,
wherein, the light conversion agent is a light conversion agent of claim 1.

6. The light conversion adhesive film composition according to claim 5, wherein, in terms of weight percentage, the light conversion adhesive film composition comprises:
98% to 99.98% of the matrix resin;
0.01% to 1% of the light conversion agent; and
0.01% to 1% of the auxiliary agent.

7. The light conversion adhesive film composition according to claim 5, wherein, the matrix resin is selected from any one or more of EVA, PVA, PMMA, POE, and an organic silicon.

8. The light conversion adhesive film composition according to claim 5, wherein, the auxiliary agent comprises any one of a main crosslinking agent, an auxiliary crosslinking agent, a silane coupling agent, and an inorganic powder, or a combination of at least two of the above.

9. A light conversion adhesive film obtained by mixing and molding an adhesive film composition, wherein, the adhesive film composition is the light conversion adhesive film composition of claim 5.

10. A photovoltaic module comprising a light conversion adhesive film, wherein, the light conversion adhesive film is the light conversion adhesive film of claim 9.

11. The light conversion agent according to claim 2, wherein, the R₁ is selected from any one of a substituted or unsubstituted C₂-C₁₀ linear alkyl, a substituted or unsubstituted C₃-C₁₅ branched alkyl, a substituted or unsubstituted C₂-C₁₀ heteroalkyl, a substituted or unsubstituted C₂-C₁₀ alkenyl, a substituted or unsubstituted C₂-C₁₀ ester group, a C₁-C₁₀ hydrocarbyl substituted or unsubstituted amino, a C₆-C₂₀ aryl substituted or unsubstituted amino, a C₁-C₁₀ hydrocarbylsubstituted or unsubstituted amido, a substituted or unsubstituted C₃-C₁₀ cyclic amido, a substituted or unsubstituted C₃-C₁₀ cyclic imide group, a C₁-C₁₀ hydrocarbyl substituted or unsubstituted carboxyl, a C₁-C₁₀ hydrocarbyl substituted or unsubstituted carbonyl, and a C₁-C₁₀ hydrocarbyl substituted or unsubstituted hydroxyl; and
the R₂, the R₃ and the R₄ are each independently selected from any one of H, a substituted or unsubstituted C₂-C₁₀ linear alkyl, a substituted or unsubstituted C₃-C₁₅ branched alkyl, a substituted or unsubstituted C₂-C₁₀ heteroalkyl, a substituted or unsubstituted C₂-C₁₀ alkenyl, a substituted or unsubstituted C₆-C₃₀ aryl, a substituted or unsubstituted C₄-C₂₀ heteraryl, a substituted or unsubstituted C₂-C₁₀ ester group, a C₁-C₁₀ hydrocarbyl substituted or unsubstituted amino, a C₆-C₂₀ aryl substituted or unsubstituted amino, a C₁-C₁₀ hydrocarbyl substituted or unsubstituted amido, a substituted or unsubstituted C₃-C₁₀ cyclic amido, a substituted or unsubstituted C₃-C₁₀ cyclic imide group, a C₁-C₁₀ hydrocarbyl substituted or unsubstituted carboxyl, a C₁-C₁₀ hydrocarbyl substituted or unsubstituted carbonyl, and a C₁-C₁₀ hydrocarbyl substituted or unsubstituted hydroxyl.

12. The light conversion agent according to claim 3, wherein, the R₁, the R₂, the R₃ and the R₄ are each independently selected from any one of the following substituted or unsubstituted substituents: n-hexyl, n-heptyl, n-octyl, n-nonyl, n-decyl, isopropyl, isobutyl, isopentyl, isohexyl, isoheptyl, isooctyl, isononyl, isodecyl, cyclopropyloxy, cyclobutoxy, cyclopentoxy, cyclohexyloxy, hexyloxy, heptyloxy, octyloxy, nonyloxy, decyloxy, ethenyl, propenyl, butenyl, pentenyl, hexenyl, heptenyl, octenyl, nonenyl, decenyl, allyl, phenyl, naphthyl, biphenyl, furyl, thienyl, indolyl, pyridyl, benzofuryl, benzothiophenyl, methyl formate group, ethyl formate group, ethyl propionate group, butyl propionate group, ethyl butyrate group, butyl butyrate group, methyl hexanoate group, methyl heptanoate group, ethyl caprylate group, amino, formamido, acetamido, propionamido, butyramido, pentanamido, cyclopropionamido, cyclobutyramido, cyclopropanoylimino, cyclobutanoylimino, carboxyl, or carbonyl.

13. The light conversion agent according to claim 11, wherein, the R₁, the R₂, the R₃ and the R₄ are each independently selected from any one of the following substituted or unsubstituted substituents: n-hexyl, n-heptyl, n-octyl, n-nonyl, n-decyl, isopropyl, isobutyl, isopentyl, isohexyl, isoheptyl, isooctyl, isononyl, isodecyl, cyclopropyloxy, cyclobutoxy, cyclopentoxy, cyclohexyloxy, hexyloxy, heptyloxy, octyloxy, nonyloxy, decyloxy, ethenyl, propenyl, butenyl, pentenyl, hexenyl, heptenyl, octenyl, nonenyl, decenyl, allyl, phenyl, naphthyl, biphenyl, furyl, thienyl, indolyl, pyridyl, benzofuryl, benzothiophenyl, methyl formate group, ethyl formate group, ethyl propionate group, butyl propionate group, ethyl butyrate group, butyl butyrate group, methyl hexanoate group, methyl heptanoate group, ethyl caprylate group, amino, formamido, acetamido, propionamido, butyramido, pentanamido, cyclopropionamido, cyclobutyramido, cyclopropanoylimino, cyclobutanoylimino, carboxyl, or carbonyl.

14. The light conversion agent according to claim 2, wherein, the naphthotriazole compound has the structural general formula as shown as follows: 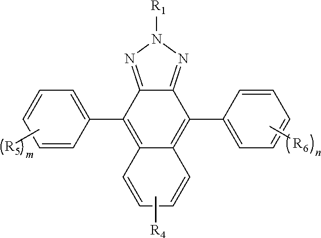
wherein, m and n are each independently 1, 2, 3, or 4;
the R₁ is selected from any one of a substituted or unsubstituted C₁-C₁₂ linear alkyl, a substituted or unsubstituted C₃-C₁₀ branched alkyl, a substituted or unsubstituted C₂-C₁₆ alkenyl, a substituted or unsubstituted C₃-C₈ ester group, a C₅-C₈ hydrocarbyl substituted or unsubstituted amino, a C₆-C₁₀ aryl substituted or unsubstituted amino, a C₅-C₈ hydrocarbyl substituted or unsubstituted amido, a substituted or unsubstituted C₅-C₈ cyclic amido, a substituted or unsubstituted C₅-C₈ cyclic imide group, a C₅-C₈ hydrocarbyl substituted or unsubstituted carboxyl, a C₅-C₈ hydrocarbyl substituted or unsubstituted carbonyl, and a C₅-C₈ hydrocarbyl substituted or unsubstituted hydroxyl;
preferably, the R₄, R₅ and R₆ are each independently selected from any one of H, a substituted or unsubstituted C₁-C₁₂ linear alkyl, a substituted or unsubstituted C₃-C₁₀ branched alkyl, a substituted or unsubstituted C₂-C₁₆ alkenyl, a substituted or unsubstituted C₃-C₈ ester group, a C₅-C₈ hydrocarbyl substituted or unsubstituted amino, a C₆-C₁₀ aryl substituted or unsubstituted amino, a C₅-C₈ hydrocarbyl substituted or unsubstituted amido, a substituted or unsubstituted C₅-C₈ cyclic amido, a substituted or unsubstituted C₅-C₈ cyclic imide group, a C₅-C₈ hydrocarbyl substituted or unsubstituted carboxyl, a C₅-C₈ hydrocarbyl substituted or unsubstituted carbonyl, and a C₅-C₈ hydrocarbyl substituted or unsubstituted hydroxyl; preferably, the R₄ is H.

15. The light conversion agent according to claim 4, wherein, the R₁, the R₅ and the R₆ are each independently selected from any one or more of a substituted or unsubstituted C₅-C₈ linear alkyl, a substituted or unsubstituted C₆-C₁₀ branched alkyl, a substituted or unsubstituted C₅-C₈ alkenyl, a substituted or unsubstituted C₃-C₈ ester group, and a phenyl substituted or unsubstituted amino, further, preferably, the R₁, the R₅ and the R₆ are each independently selected from any one of 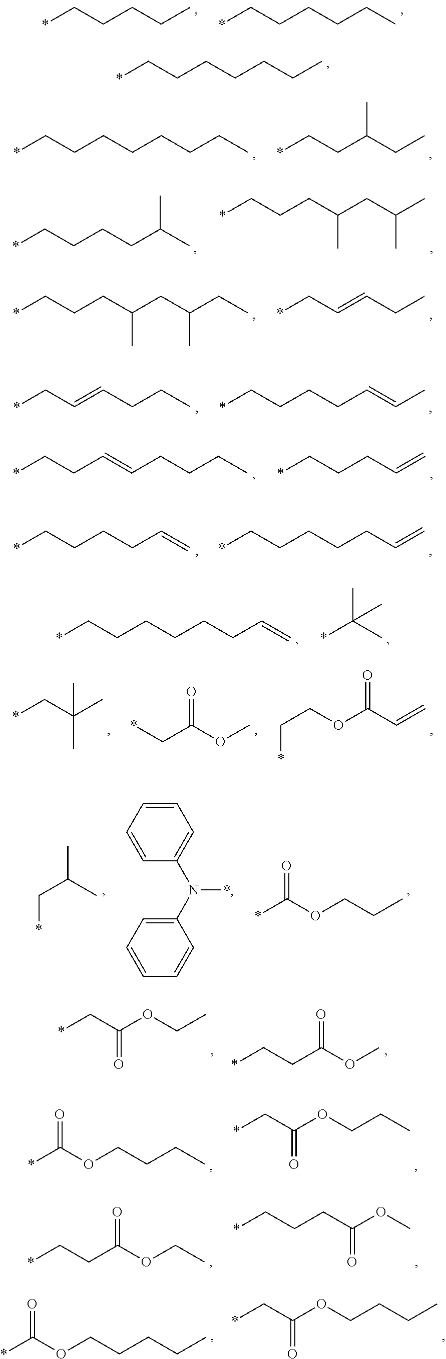 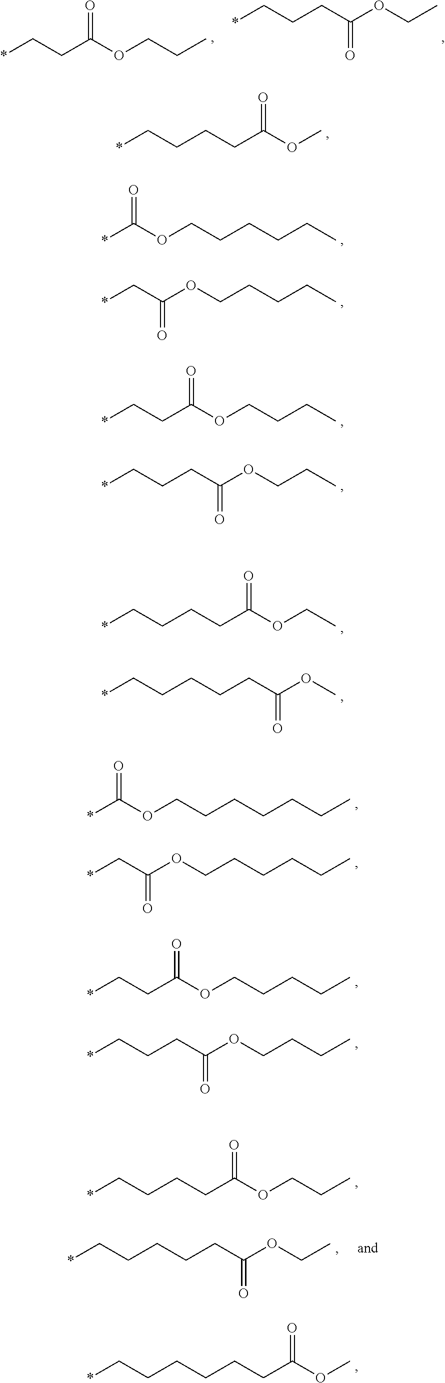
wherein, “*” indicates the attachment position of the substituent on the naphthotriazoles.

16. The light conversion agent according to claim 14, wherein, the R₁, the R₅ and the R₆ are each independently selected from any one or more of a substituted or unsubstituted C₅-C₈ linear alkyl, a substituted or unsubstituted C₆-C₁₀ branched alkyl, a substituted or unsubstituted C₅-C₈ alkenyl, a substituted or unsubstituted C₃-C₈ ester group, and a phenyl substituted or unsubstituted amino, further, preferably, the R₁, the R₅ and the R₆ are each independently selected from any one of 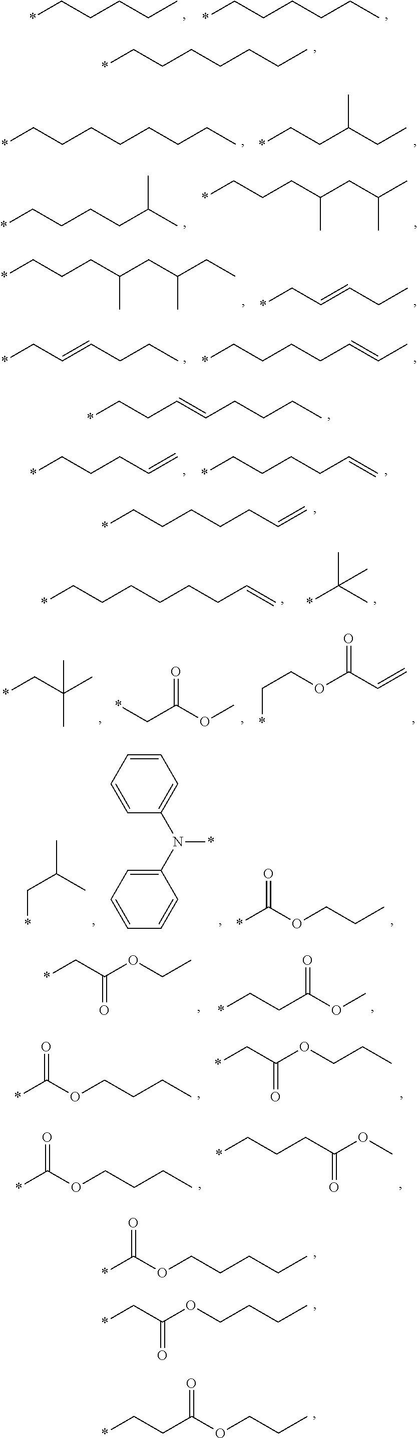 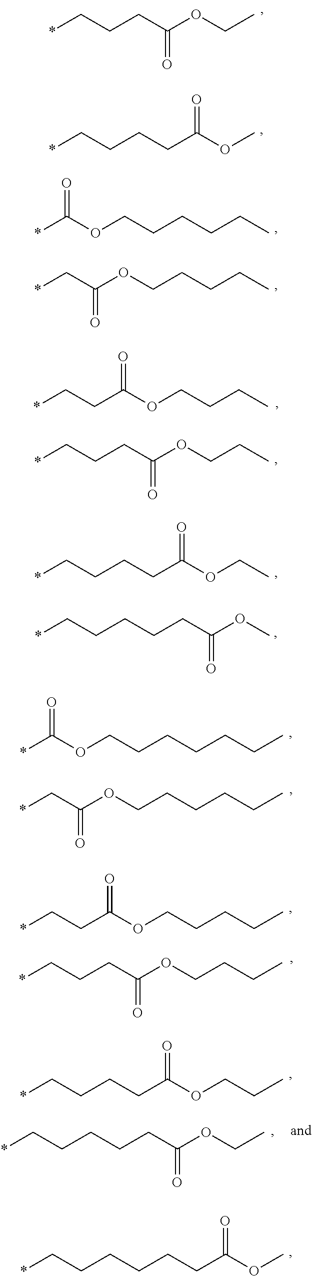
wherein, “*” indicates the attachment position of the substituent on the naphthotriazoles.

17. The light conversion agent according to claim 15, wherein, the R₁ is selected from any one of 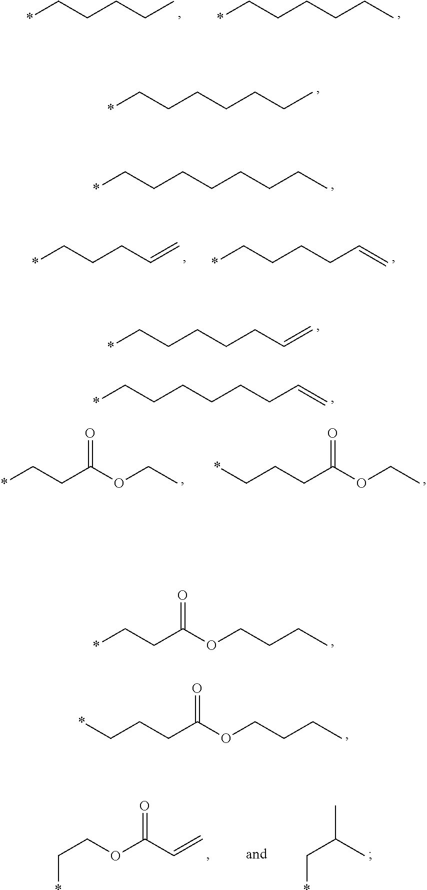
preferably, the R₅ and the R₆ are each independently selected from any one of 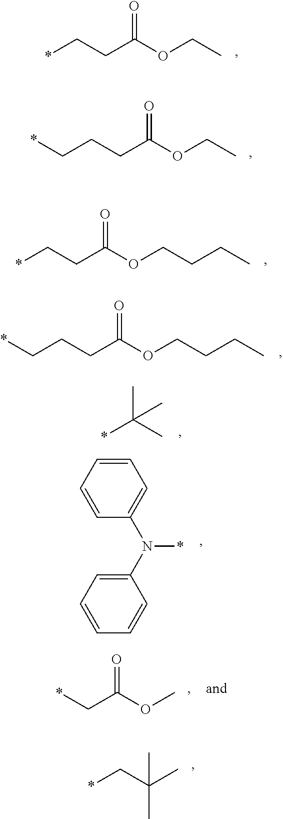
further, preferably, the R₅ and the R₆ are simultaneously any one of 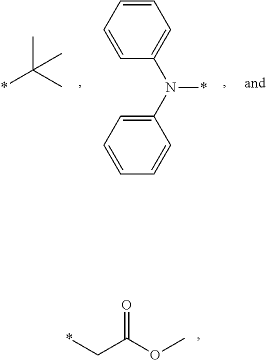
wherein, “*” indicates the attachment position of the substituent on the naphthotriazoles.

18. The light conversion agent according to claim 16, wherein, the R₁ is selected from any one of 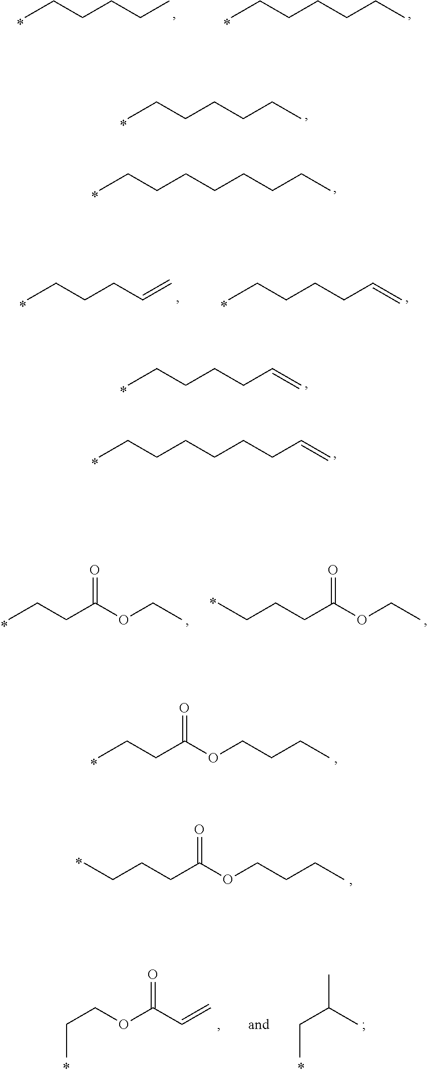
preferably, the R₅ and the R₆ are each independently selected from any one of 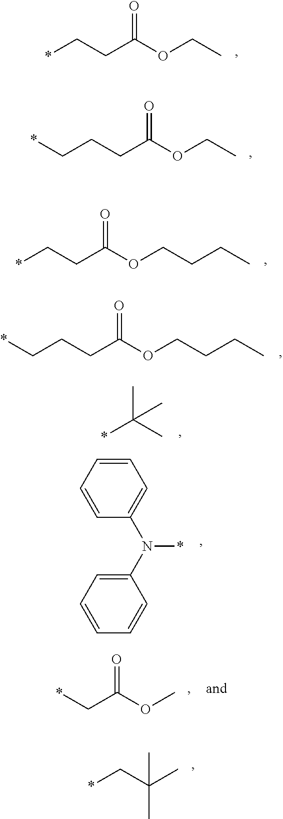
further, preferably, the R₅ and the R₆ are simultaneously any one of 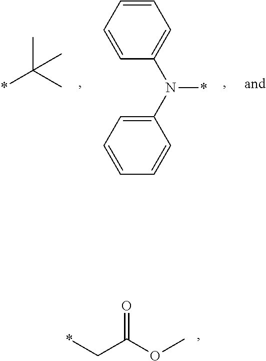
wherein, “*” indicates the attachment position of the substituent on the naphthotriazoles.

19. The light conversion adhesive film composition according to claim 6, wherein the mass ratio of the light conversion agent to the auxiliary agent is 1:2-500.

20. The light conversion adhesive film composition according to claim 19, wherein the mass ratio of the light conversion agent to the auxiliary agent is 1:2-50.
